(19) United States
(12) Patent Application Publication
(10) Pub. No.: US 2023/0227781 A1
(43) Pub. Date: Jul. 20, 2023

(54) METHOD OF PRODUCING MACROPHAGES

(71) Applicant: The University Court of The University of Edinburgh, Edinburgh (GB)

(72) Inventors: Stuart Forbes, Edinburgh (GB); Lara Campana, Edinburgh (GB); Benjamin Dwyer, Edinburgh (GB); John Campbell, Edinburgh (GB); Alasdair Fraser, Edinburgh (GB)

(21) Appl. No.: 17/927,786

(22) PCT Filed: May 27, 2021
(86) PCT No.: PCT/GB2021/051294

§ 371 Date: Nov. 25, 2022

(30) Foreign Application Priority Data
May 27, 2020 (GB) 2007903.4

(51) Int. Cl. C12N 5/0786; A61K 35/15

(52) U.S. Cl. CPC C12N 5/0645; A61K 35/15; C12N 2506/115; C12N 2501/22; C12N 2501/2304; C12N 2501/231; C12N 2501/2313; C12N 2501/24

(57) ABSTRACT

The improved 4-5 day, optionally 3-5 day GMP-compliant in-vitro method enables the production of macrophages from monocytes that benefits from a shorter cell culture time, fewer interventions whilst maintaining the desired characteristics of the human macrophages. The present invention describes a method wherein the monocytes are cultured in medium comprising one or more growth actors to stimulate macrophages with a pro-regenerative phenotype. The method described herein is xeno-free, serum-free and GMP compliant. In addition, further disclosed are macrophages produced according to the present invention and the use of said macrophages in the treatment of liver diseases, such as liver cirrhosis.

### TECHNICAL FIELD

The present invention relates to a 4-5 day, optionally a 3-5 day GMP-compliant method for producing mature macrophages, in particular monocyte derived macrophages, and to the macrophages produced by the method. The invention further relates to medical uses of said macrophages, in particular the treatment of liver disease or injury.

### BACKGROUND

Fibrosis is the final common pathway of chronic liver disease of various aetiologies, including toxic damage, viral infections, metabolic and genetic diseases, and autoimmune hepatitis. Whereas acute, self-limiting fibrosis has likely evolved as a reversible and protective response to liver injury. The balance between self-limited and excessive fibrosis is finely regulated by multiple pathways and systems, and essentially dependant on the duration and repetition of the injury. End-stage, chronic liver fibrosis also known as cirrhosis, is a life-threatening condition (1-3). Mortality due to liver disease is the only leading cause of death that has increased year on year since the 1970s in the United Kingdom and it remains a major health burden worldwide. The only therapeutic approaches entail removal of the injurious stimuli (e.g. administration of an efficacious anti-viral therapy) and liver transplantation. Delivering an effective anti-fibrotic therapy is therefore a major unmet clinical need for both chronic and acute liver damage (4-6).

Macrophages (Mϕ) play a pivotal role in the inflammatory response in the injured liver. In the liver there are two main populations of Mϕ: (i) the resident macrophages (KCs), and (ii) the infiltrating macrophages. KCs carry out patrolling functions in the liver sinusoids to phagocytose microbial debris that reach the liver via the sinusoidal capillaries in homeostatic conditions. During the early phases of liver damage, KCs express chemokines such as CCL2 and CCL5, thereby contributing to the recruitment of monocytes from the circulation (2, 7). The number of KCs decreases during fibrosis; they then repopulate the liver in the recovery phase of self-limiting fibrosis (8). Infiltrating, monocyte derived Mϕ play a major role in the response to liver damage. Infiltrating Mϕ are recruited via the CCR2/CCL2 axis; once in the liver parenchyma, they locate along the fibrotic septa in the early stages of liver fibrosis and may promote fibrosis by releasing factors such as TGF-β, IL1, PDGF and CCL2 that activate hepatic stellate cells and worsen inflammation. This may suggest a detrimental role for Mϕ in progressive fibrosis. However, if Mϕ are depleted at the onset of fibrosis remodelling, the remodelling process fails, and the liver fibrosis persists. It is now widely accepted that macrophages play a dual role in the establishment and resolution of fibrosis (2, 8-10).

Due to their role in healing of fibrosis, it has been considered that a macrophage cell therapy could be beneficial for the reduction of chronic liver fibrosis. It has been shown that mouse bone marrow-derived macrophages (BMDMs) improve liver fibrosis when injected into a mouse model of chronic liver fibrosis (11). Similar results have been replicated using human monocyte-derived macrophages (hMDMs) in immunodeficient mouse models of chronic liver fibrosis (12). Furthermore, a GMP (good manufacturing practice)-graded cell culture protocol (13) is currently used to generate hMDMs for autologous transplantation into cirrhotic patients in an ongoing phase II trial (Macrophage therapy for liver disease, ISRCTN10368050). Good manufacturing practice (GMP) quality, defined by both the European Medicines Agency (EMA) and the Food and Drug Administration (FDA), is a requirement for clinical-grade cells, offering optimal defined quality and safety in cell therapy. Using animal component-free culture media and compliant cryopreservation procedures, immune reactions against animal proteins and infection risk caused by animal microbes can be avoided. In the development of cell therapies, compliant procedures must be developed to ensure the safe preparation and storage of cells prior to administration to a patient in need thereof.

However, the protocols used to generate these hMDMs are slow and laborious. Currently, hMDMs are differentiated from circulating monocytes by culturing them for seven days in high-dose MCSF (Monocyte Colony Stimulating Factor, also known as CSF1, Colony Stimulating Factor 1), with a feed of fresh medium and MCSF at various time points (13, 14). It would be desirable to reduce the time and effort taken to culture hMDMs. However this far, it has been considered unfeasible to do so. There are a number of hurdles to overcome in the manufacturing process to culture and produce cells in accordance with the GMP guidelines (Giancola, R., Bonfini, T., & Iacone, A. (2012). Cell therapy: cGMP facilities and manufacturing. Muscles, ligaments and tendons journal, 2(3), 243-247.). Changing a cell culture protocol that is routinely used in laboratory experiments to a GMP-compliant method suitable for clinical applications is often met with challenges affecting the scalability, yield, morphology and function of the cells. Attempts to shorten and simplify such methods of production have failed to reproduce mature hMDMs which are suitable for GMP-compliant therapies and which retain the desired characteristics of hMDMs for therapeutic use.

The present inventors sought to solve the above-mentioned problem by providing a second-generation method which would benefit from both shortening the cell culture time and eliminating intermediate feeding steps whilst maintaining the required characteristics of hMDMs for therapeutic use. Advantageously, this improved method is xeno-free, serum-free and GMP-compliant, making the cells suitable for clinical use.

One or more aspects of the invention are directed towards solving one or more of the above-mentioned problems.

### STATEMENTS OF INVENTION

According to a first aspect of the present invention, there is provided an in vitro method of producing macrophages comprising:
(a) Culturing monocytes in medium for 4-5 days to produce macrophages, wherein the medium comprises one or more growth factors to stimulate macrophage production.

According to an alternative first aspect of the present invention, there is provided an in vitro method of producing macrophages comprising:
(a) Culturing monocytes in medium for 4-5 days to produce macrophages, wherein the medium comprises one or more growth factors to stimulate macrophage production;
Wherein step (a) takes place entirely in the same medium.

According to another aspect of the present invention, there is provided an in vitro method of producing macrophages comprising:
(b) Culturing monocytes in medium for 3-5 days to produce macrophages, wherein the medium comprises one or more growth factors to stimulate macrophage production.

According to another first aspect of the present invention, there is provided an in vitro method of producing macrophages comprising:
(b) Culturing monocytes in medium for 3-5 days to produce macrophages, wherein the medium comprises one or more growth factors to stimulate macrophage production;
Wherein step (a) takes place entirely in the same medium.

According to a second aspect of the present invention, there is provided a macrophage produced by the method of the first aspect.

According to a third aspect of the present invention, there is provided an ex vivo generated macrophage having a pro-regenerative phenotype.

In one embodiment, the ex vivo generated macrophage is unpolarised. In one embodiment, the ex vivo generated macrophage is an unpolarised mature macrophage. In one embodiment, the ex vivo generated macrophage has an anti-inflammatory phenotype. In one embodiment, the ex vivo generated macrophage has an anti-fibrogenic phenotype. In one embodiment, the ex vivo generated macrophage has an anti-inflammatory and anti-fibrogenic phenotype.

According to a fourth aspect of the present invention, there is provided a population of macrophages according to the second or third aspects.

According to a fifth aspect of the present invention, there is provided a composition comprising a macrophage according to the second or third aspects, or a population according to the fourth aspect.

In one embodiment, the composition is a pharmaceutical composition.

According to a sixth aspect of the present invention, there is provided a cell culture bag comprising macrophages of the second or third aspects, a composition of the fifth aspect, or a population of macrophages of the fourth aspect.

According to a seventh aspect of the present invention, there is provided a macrophage according to the second or third aspects, or a composition according to the fifth aspect for use as a medicament.

According to an alternative seventh aspect of the present invention, there is provided a method of treatment of a subject having a disease by administering an effective amount of macrophages according to the second or third aspects, or a composition according to the fifth aspect, to the subject.

According to an eighth aspect of the present invention, there is provided a macrophage according to the second or third aspects, or a composition according to the fifth aspect, for use in the treatment of liver disease or injury.

According to an alternative eighth aspect of the present invention, there is provided a method of treatment of a subject having a liver disease or injury, comprising administering an effective amount of macrophages according to the second or third aspects, or a composition according to the fifth aspect, to the subject.

According to a ninth aspect there is provided use of macrophages according to the second or third aspects, or a composition according to the fifth aspect, in the manufacture of a medicament for treating a disease in a subject, the manufacture comprising the steps of:
(a) Culturing monocytes in medium for 4-5 days to produce macrophages, wherein the medium comprises one or more growth factors to stimulate macrophage production;
and
(b) Formulating some or all of said macrophages into a medicament for administration to the subject.

According to an alternative ninth aspect there is provided use of macrophages according to the second or third aspects, or a composition according to the fifth aspect, in the manufacture of a medicament for treating a disease in a subject, the manufacture comprising the steps of:
(a) Culturing monocytes in medium for 4-5 days to produce macrophages, wherein the medium comprises one or more growth factors to stimulate macrophage production;
and
(b) Formulating some or all said macrophages into a medicament for administration to the subject,
Wherein step (a) takes place entirely in the same medium.

According to a different ninth aspect there is provided use of macrophages according to the second or third aspects, or a composition according to the fifth aspect, in the manufacture of a medicament for treating a disease in a subject, the manufacture comprising the steps of:
(c) Culturing monocytes in medium for 3-5 days to produce macrophages, wherein the medium comprises one or more growth factors to stimulate macrophage production;
and
(d) Formulating some or all of said macrophages into a medicament for administration to the subject.

According to a another ninth aspect there is provided use of macrophages according to the second or third aspects, or a composition according to the fifth aspect, in the manufacture of a medicament for treating a disease in a subject, the manufacture comprising the steps of:
(c) Culturing monocytes in medium for 3-5 days to produce macrophages, wherein the medium comprises one or more growth factors to stimulate macrophage production;
and
(d) Formulating some or all said macrophages into a medicament for administration to the subject,
Wherein step (a) takes place entirely in the same medium.

In preferred embodiments, the disease is a liver disease or injury.

Advantageously, the inventors have discovered a novel GMP-compliant method of producing macrophages from monocytes which is several days shorter than prior art methods, and which does not require any re-feeding of medium. Conversely to what is commonly reported in literature, the macrophages produced according to the method herein show characteristics of mature macrophages as early as three days after the start of the culture, and do not require any re-feeding of medium. Advantageously, the key markers that are characteristics of the desirable macrophages such as 25F9 and CD206 are up-regulated as early as 3 days of culture (FIGS. 4B & 4C). Markers that are typically expressed on monocytes, such as CCR2, are down-regulated as early as 3 days of culture (FIG. 4F). This demonstrates the production of macrophages at 3 days of culturing monocytes.

Therefore, the method of the invention is quicker than previous methods and is GMP (Good Manufacturing Practice)-compliant. Furthermore, the method of the invention has been found to have a higher yield of macrophages than prior art methods.

The inventors have surprisingly found that the macrophages produced by the novel method also have a different cytokine profile and expression marker profile than those produced by other methods. The macrophages have a pro-regenerative profile compared to macrophages produced with the current state of the art protocol. Further, these macrophages have a superior ability to respond to polarising stimuli such as LPS, IFNγ, IL4-IL13 in combination and IL10. They also have lower levels of adhesion to plastic surfaces, making them easier to transport and deliver.

Finally, the inventors have shown that the macrophages described herein are able to treat acute and chronic conditions as indicated by in vivo testing in models of paracetamol (acetaminophen, APAP) overdose and CCl₄-induced liver cirrhosis in mice.

Further aspects of the invention are defined as follows:

In a further aspect there is provided a composition or a population of macrophages having reduced expression of one or more of the following inflammatory cytokines: IL1, IL12, IL17 (A, B, C, F), IL18, TNFα, IFNγ.

In a further aspect there is provided a composition or population of macrophages that express less than 200 pg/mL of IL17F, suitably less than 190 pg/mL of IL17F, less than 180 pg/mL of IL17F, suitably less than 170 pg/mL of IL17F, suitably less than 160 pg/mL of IL17F, suitably 150 pg/mL or less of IL17F.

In a further aspect there is provided a composition or population of macrophages that express less than 45000 pg/mL of TGFβ, suitably less than 44000 pg/mL, suitably less than 43000 pg/mL, suitably less than 42000 pg/mL, suitably less than 41000 pg/mL, suitably 40000 pg/mL or less of TGFβ.

In a further aspect there is provided a composition or population of macrophages that express more than 50 pg/mL VEGFR1, more than 100 pg/mL of VEGFR1, more than 120 pg/mL of VEGFR1, more than 140 pg/mL VEGFR1, more than 160 pg/mL VEGFR1, more than 170 pg/mL of VEGFR1.

In a further aspect there is provided a composition or population of macrophages that express less than 500 pg/mL of IL9, less than 300 pg/mL of IL9, less than 200 pg/mL IL9, less than 180 pg/mL IL9, less than 160 pg/mL IL9, less than 140 pg/mL IL9, less than 130 pg/mL of IL9.

In a further aspect there is provided a composition or population of macrophages that possess an anti-inflammatory and anti-fibrogenic phenotype, wherein the macrophages express less than 200 pg/mL of IL17F and less than 45000 pg/mL of TGFβ.

In a further aspect there is provided a composition or population of ex vivo generated macrophages in which the macrophages have an anti-inflammatory and anti-fibrogenic phenotype, wherein the macrophages express around 150 pg/mL of IL17F and around 40000 pg/mL of TGFβ.

In a further aspect there is provided a composition or population of macrophages that are mature macrophages, wherein the mature macrophages are CD14+, CD206+, CD163+, CD169+, 25F9+, and CD86+, and wherein the mature macrophages are CCR2−.

In a further aspect there is provided a composition or population of macrophages which respond to inflammatory stimuli such as one or more of: IFNγ, IL10, IL4, IL13, and LPS.

In a further aspect there is provided a composition or population comprising a yield of cultured mature macrophages produced by the method of the first aspect of at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38, up to 40%, up to 45%, up to 50%.

In an embodiment of any of the above aspects, the level of cytokine expression from macrophages is measured by testing the cell culture supernatant using V-plex technology as explained in the examples.

Further features and embodiments of the present invention will now be described. Features are not restricted to any particular aspect or embodiment of the invention and may be combined in any compatible way.

### DESCRIPTION

The following definitions are provided.

‘hMDM’ as used in the present invention refers to human monocyte-derived macrophages. Monocyte-derived means macrophages differentiated from monocytes. Monocytes are the natural precursors of macrophages and dendritic cells; they are contained in blood and bone marrow.

‘unpolarised macrophage’ as used in the present invention refers to a mature macrophage which has not received any further stimulation to induce specific functional capacity, unpolarised macrophages may also refer to naïve or non-activated macrophages.

‘polarised macrophage’ as used in the present invention refers to a macrophage which has received environmental stimulus to become activated into a particular phenotype such as the M1-like or M2-like phenotype. The M1-like and M2-like phenotypes are described hereinbelow.

‘macrophage’ refers to a phagocytic cell which is responsible for detecting, engulfing and destroying pathogens, damaged and apoptotic cells, and which is produced through the differentiation of monocytes. The term ‘macrophage’ as used herein generally refers to a macrophage produced by the process described herein. It may be polarised or unpolarised.

‘mature macrophage’ refers to a macrophage which expresses mature cell surface markers, preferably CCR2−, CD14+, CD206+, CD163+, CD169+, 25F9+, and CD86+.

An ‘M1 polarising factor’ as used in the present invention refers to a factor which stimulates an unpolarised macrophage into an M1-like phenotype, and may refer to one or more of: GM-CSF, IFNγ, and TLR agonists, such as LPS, for example.

An ‘M2 polarising factor’ as used in the present invention refers to a factor which stimulates an unpolarised macrophage into an M2-like phenotype, and may refer to one or more of: IL10, IL4, IL13, and poly(I:C), for example.

‘GMP-compliant’ as used in the present invention means that the method complies with good manufacturing practice. By way of example a GMP-compliant medium has to be serum-free, antibiotic-free and xenoprotein-free (animal substance free). The WHO provides guidance on what is required for good manufacturing practice: “Chapter 1: WHO good manufacturing practices: Main principles for pharmaceutical products”. Quality Assurance of Pharmaceuticals: A compendium of guidelines and related materials—Good manufacturing practices and inspection. 2 (2nd updated ed.). WHO Press. pp. 17-18. ISBN 9789241547086.

‘treatment’ as used in the present invention means an intervention in a physiological condition which prevents, reduces or removes the clinical symptoms associated with a given physiological condition in a subject.

By ‘subject’ or ‘individual’ or ‘animal’ or ‘patient’ is meant any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on.

‘day 5 no feed’ or ‘day 5’ as used in the present invention in relation to a method/protocol refers to the method/protocol of the invention to produce macrophages. As defined in the first aspect, the method of the invention lasts between 3-5 days, optionally 4-5 days. 3-5 days typically refers to a period of about 72-120 hours. 4-5 days typically refers to a period of about 96-120 hours. This period may vary by +/−10 hours, preferably +/−5 hours, preferably +/−2 hours. Suitably therefore, the method of the invention may last between 62 and 130 hours, suitably between 86 and 130 hours, suitably between 90 and 125 hours suitably between 96 and 120 hours.

‘day 7 plus re-feed’ or ‘day 7’ as used in the present invention in relation to a method/protocol refers to the standard longer method/protocol currently used in the art to produce macrophages. These prior methods last for a period of around 7 days which typically refers to a period of about 168 hours. This period may vary by +/−10 hours, preferably +/−5 hours, preferably +/−2 hours.

It is to be noted that the term “a” or “an” entity refers to one or more of that entity.

‘about’ means +/−10% +/−9%, +/−8%, +/−7%, +/−6%, +/−5%, +/−4%, +/−3%, +/−2%, +/−1%, of the value given unless otherwise stated.

‘increased expression’ refers to at least 2-fold increased expression. The fold increase is calculated dividing the mean fluorescence intensity (MFI) of a given marker in the macrophages by the MFI of the same markers in the monocytes from which the macrophages have been differentiated. The ratio between the two values is herein defined as ‘MFI ratio’ or ‘Relative Fluorescence Intensity (RFI)’. These values are measured by flow cytometry. ‘increased expression’ may also refer to the increased concentration of a given cytokine in the medium of one sample as compared to another sample. The increase is considered significant when the statistical test to compare the two groups of samples returns a value <0.05.

‘decreased expression” refers to at least a RFI<0.5. The decrease is calculated dividing the MFI of a given marker in the macrophages by the MFI of the same markers in the monocytes from which the macrophages have been differentiated. The ratio between the two values is herein defined as ‘MFI ratio’ or ‘Relative Fluorescence Intensity (RFI)’. These values are measured by flow cytometry. ‘decreased expression’ may also refer to the increased concentration of a given cytokine in the medium of one sample as compared to another sample. The decrease is considered significant when the statistical test to compare the two groups of samples returns a value <0.05.

### Cell Culture Medium

The present invention relates to a novel method for producing macrophages from monocytes, in which the monocytes are cultured in medium for 3-5 days, and in which the same medium is used throughout this step.

Suitably the medium is suitable for generating macrophages from monocytes. Suitably the medium is a T-cell medium. Suitably the medium may be selected from: X-Vivo 10, X-Vivo 15, TexMACS, AIMv, RPMI, DMEM, and DMEM/F12. Suitably the medium is TexMACS (Miltenyi).

Suitably the medium is serum-free. Suitably the medium is xenoprotein-free. Suitably the medium is GMP-compliant.

Suitably the medium may contain one or more factors. Suitable factors include growth factors, polysaccharides, cytokines and chemokines. Suitable factors may include: MCSF, GM-CSF. Suitably therefore the factors are growth factors. Suitably, the one or more factors are GMP-compliant. In one embodiment, the medium may comprise one or more growth factors may include MCSF or GM-CSF. Monocytes are most commonly cultured with either MCSF or GM-CSF. Culturing monocytes with GM-CSF skews them towards an “M1-like phenotype” whereas culturing monocytes with MCSF skews them towards an “M2-like phenotype”. However, culturing monocytes with both MCSF and GM-CSF is an unorthodox approach. In other embodiments, the one or more growth factors does not include the combination of MCSF and GM-CSF. Thus, if M-CSF is used as the growth factor to generate macrophages in any of the methods of the invention, it may be preferred that GM-CSF is not also used to generate the macrophages from monocytes. This applies to the step of culturing monocytes until macrophages are generated.

Suitably the medium contains MCSF (macrophage colony stimulating factor) otherwise known as CSF-1. Suitably the MCSF may be recombinant MCSF, suitably recombinant human MCSF.

Suitably the medium contains MCSF at a concentration of between 25-200 ng/mL, suitably at a concentration of between 50-125 ng/mL, suitably at a concentration of between 75-110 ng/mL suitably at a concentration of 100 ng/mL (EQUIVALENT IN INTERNATIONAL UNIT (IU): 100 ng of recombinant human GMP-graded MCSF=1.6×10⁴ IU).

Suitably the one or more factors are added to the medium. Suitably the methods may comprise a step of adding the one or more factors to the medium. Suitably adding the one or more factors may take place before step (a) and/or during step (a).

Suitably MCSF is added to the medium. Suitably the methods of the invention comprise a step of adding MCSF to the medium. Suitably MCSF is added to the medium before culturing step (a).

Suitably the medium may contain one or more further additives. Suitable further additives may include albumin, glutamine, and antibiotics such as streptomycin or penicillin.

Suitably the medium may contain one or more indicators, such as a pH indicator, for example.

Suitably the one or more further additives are added to the medium, alternatively the medium may already comprise said further additives. Suitably the methods may comprise a step of adding the one or more further additives to the medium. Suitably adding the one or more further additives may take place before step (a) and/or during step (a).

In one embodiment, the medium is free from additives.

Suitably at least step (a) takes place in entirely the same medium. Suitably the differentiation of monocytes into macrophages takes place in the same medium. Suitably the whole method takes place in the same medium. Suitably by ‘the same medium’ it is meant that no addition, re-feeding or replacement of medium takes place, i.e. this is a single-feed method. Methods that are commonly reported in literature are often ‘multiple-feed methods’ i.e. they involve additions, re-feeding or replacement of medium. The disadvantage with ‘multiple-feed methods’ is that cells that are not attached to the surface of the culture plate are often removed in the process of replacing medium. In other cases, addition of components or re-feedings can often disrupt cells growing in the culture plate. The more intervention involved in a process, the higher the loss of cells during that process. Advantageously, the present method involves a single-feed and therefore involves fewer interventions and thus results in fewer cells lost. Suitably step (a) does not comprise re-feeding of medium. Suitably step (a) does not comprise replacing the medium. Suitably the method does not comprise a step of re-feeding medium. Suitably the method does not comprise a step of replacing the medium.

### Culturing Method

The present invention relates to a novel method for culturing monocytes to produce macrophages.

Suitably the methods may further comprise a step of obtaining monocytes. Suitably monocytes may be obtained from peripheral blood or leukapheresis collection or mobilised apheresis collection (such as M-CSF, GM-CSF or G-CSF mobilised blood precursors). Suitably from human blood, suitably a human blood sample. Suitably the monocytes are obtained from the mononuclear leukocyte fraction of human blood, suitably from the mononuclear leukocyte fraction of a human blood sample.

Suitably the methods may further comprise a step of purifying monocytes from blood, suitably from the mononuclear leukocyte fraction of blood, suitably from the mononuclear leukocyte fraction of a human blood sample. Such purification may comprise isolation of the mononuclear leukocyte fraction, and isolation of purified monocytes from the fraction using specific (markers of monocyte lineage) or non-specific (adherence) methods. Suitably isolation of the mononuclear leukocyte fraction may be carried out by various methods depending upon source material. Suitably isolation of selected purified monocytes may be carried out at small scale (magnetic bead column devices or plastic adherence) or at larger scale for manufacturing using relevant devices such as the CliniMACS Prodigy system (Miltenyi Biotec). In other embodiments, the method may further comprise a step of administering a mobilising drug to the patient prior to obtaining an apheresis collection. Suitably, the mobilising drugs encourages precursor cells to mobilise from the bone marrow.

Suitably the methods comprise a step of culturing isolated monocytes in medium for 3-5 days to produce macrophages. Suitably the monocytes may be cultured in medium within plates or within cell culture cell culture bags as further described below.

Suitably the monocytes are seeded at a density of 1×10⁶ cells/cm² up to 1×10⁸ cells/cm², suitably at a density of 5×10⁶ cells/cm² up to 5×10⁷ cells/cm², suitably at a density of 7×10⁶ cells/cm².

Suitably the monocytes are cultured in a humidified atmosphere.

Suitably the monocytes are cultured at a temperature of 35° C. to 39° C., suitably of 36° C. to 38° C., suitably at about 37° C.

Suitably the monocytes are cultured in an atmosphere comprising carbon dioxide. Suitably the carbon dioxide is at a concentration of 1-20%, suitably 2-15%, suitably 3-10%, suitably 4-8%, suitably about 5%.

Suitably the monocytes are cultured for 4-5 days, optionally 3-5 days. In one embodiment, the monocytes are cultured for 5 days. Suitably the monocytes are cultured continuously for 4-5 days, optionally for 3-5 days. In one embodiment, the monocytes are cultured continuously for 5 days.

Suitably the methods may further comprise a step of isolating mature macrophages from the medium, suitably isolating mature macrophages from the medium of step (a). Suitably the mature macrophages are isolated using typical cell dissociation techniques, for example cell dissociation buffer (Gibco, ThermoFisher) and a pastette, or automated or manual manipulation of the cell culture bag.

Suitably the methods may further comprise a step of formulating the mature macrophages as a medicament. Suitably this step may comprise resuspending the mature macrophages in excipient. Suitably the excipient may be any pharmaceutically acceptable excipient, for example saline, as discussed elsewhere herein.

Suitably cell viability during the method is at least 70% viability, at least 75% viability, at least 80% viability, at least 85% viability, at least 90% viability, at least 95% viability.

Suitably the method of the present invention produces a high yield of macrophages. Suitably the method of the present invention produces a higher yield of macrophages than the methods of the prior art. Suitably the method of the invention produces a yield of macrophages of at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%. Suitably the method of the invention produces a yield of macrophages from 20% to 70%. Suitably, the method of the invention produces a yield of up to 40%, up to 45%, up to 50%, up to 55%, up to 60%, up to 65%, up to 70%. Suitably the yield of macrophages is calculated as (Number of macrophages at harvest/number of macrophages cultured)*100.

Suitably the method is GMP-compliant.

### Polarisation

The method of producing macrophages of the invention may comprise a further step of polarisation of the macrophages. Suitably the step of polarising the macrophages takes place after step (a) but before isolation or formulation of the macrophages. Suitably any of the methods or uses of the invention may comprise such an additional step.

Suitably the step of polarising macrophages may comprise the conversion of the macrophages of step (a) into M1-like or M2-like macrophages.

In one embodiment, there is provided an in vitro method of producing macrophages comprising:
(a) Culturing monocytes in medium for 4-5 days to produce macrophages, wherein the medium comprises one or more growth factors to stimulate macrophage production; and
(b) Polarising the macrophages produced in step (a).

In one embodiment, there is provided an in vitro method of producing macrophages comprising:
(a) Culturing monocytes in medium for 4-5 days to produce macrophages, wherein the medium comprises one or more growth factors to stimulate macrophage production; and
(b) Polarising the macrophages produced in step (a).
Wherein step (a) takes place entirely in the same medium.

In another embodiment, there is provided an in vitro method of producing macrophages comprising:
(c) Culturing monocytes in medium for 3-5 days to produce macrophages, wherein the medium comprises one or more growth factors to stimulate macrophage production; and
(d) Polarising the macrophages produced in step (a).

In a further embodiment, there is provided an in vitro method of producing macrophages comprising:
(c) Culturing monocytes in medium for 3-5 days to produce macrophages, wherein the medium comprises one or more growth factors to stimulate macrophage production; and
(d) Polarising the macrophages produced in step (a).
Wherein step (a) takes place entirely in the same medium.

In a further embodiment, the one or more growth factors are GMP-graded growth factors, such as GMP GM-CSF and GMP MCSF.

Suitably the step of polarising macrophages may comprise the addition of one or more polarising factors to the medium. Suitably the one or more polarising factors may produce M1-like or M2-like macrophages. Suitably the M1-like and the M2-like phenotype are generated by polarisation with various factors as explained below.

Suitably the M1-like phenotype is pro-inflammatory.

Suitably the M2-like phenotype is pro-restorative.

Suitably in order to produce M1-like macrophages the one or more polarising factors include: GM-CSF, IFNγ and TLR agonists such as LPS. In one embodiment the M1 polarising growth factor is IFNγ or IFNγ+LPS.

Suitably in order to produce M2-like macrophages the one or more polarising factors include: IL10, IL4, IL13, and poly(I:C). In one embodiment the M2 polarising factor is IL4+IL13. Alternatively, the M2 polarising factor is IL10. Alternatively, the M2 polarising factor is poly(I:C).

Suitably the concentration of each polarising factor added to the medium is between 10-150 ng/mL, suitably between 25-125 ng/mL, suitably between 50-100 ng/mL.

Suitably the concentration of each M1 polarising factor is between 10-100 ng/mL, suitably between 20-80 ng/mL, suitably between 30 ng/mL to 60 ng/mL, suitably about 50 ng/mL. In one embodiment the M1 polarising factor is IFNγ used at a concentration of 50 ng/mL (equivalent to 0.1×10⁴ IU/mL).

Suitably the concentration of each M2 polarising factor is between 1-20 ng/mL, suitably between 5-15 ng/mL, suitably between 8 ng/mL to 12 ng/mL, suitably about 10 ng/mL. In one embodiment the M2 polarising factors are IL4 and IL13 each used at a concentration of 10 ng/mL (equivalent to 0.29×10³ IU/mL). In an alternative embodiment, the M2 polarising factor is IL10 used at concentration of 10 ng/mL (equivalent to 0.29×10³ IU/mL).

Suitably the concentration of each M1 polarising factor is about 50 ng/mL.

Suitably the concentration of each M2 polarising factor is about 10 ng/mL.

Suitably during the polarisation step, further MCSF is added to the medium.

Suitably the further MCSF is added to the medium at a concentration of between 10-150 ng/mL, suitably at between 25-125 ng/mL, suitably between 50-100 ng/mL. Suitably the further MCSF is added to the medium at a concentration of about 50 ng/mL. Suitably 100 ng of recombinant human MCSF GMP-graded =1.6×10⁴ IU.

Suitably a method comprising a step of polarising the macrophages produces polarised macrophages. Suitably a method comprising a step of polarising the macrophages produces polarised M1-like or M2-like macrophages.

In one embodiment, there is provided a polarised macrophage produced by such methods.

In one embodiment, there is provided an ex vivo generated polarised macrophage having an anti-inflammatory and anti-fibrogenic phenotype.

In one embodiment, there is provided a population of said polarised macrophages.

In one embodiment, there is provided a cell culture bag comprising said polarised macrophages or a population thereof.

Suitably the polarised macrophages may be M1-like or M2-like macrophages.

### Monocytes

The present invention relates to a novel method for culturing monocytes to produce macrophages.

Suitably, therefore, the method of the invention may further comprise a step of obtaining monocytes.

Suitably the human monocytes are provided from any source such whole blood, mononuclear cells, leukapheresis, mobilised apheresis or they may be iPSC-derived.

Suitably the monocytes are derived from human blood, suitably from the mononuclear leukocyte fraction of human blood. Suitably the monocytes are derived from a human peripheral blood or leukapheresis donation, suitably from the mononuclear leukocyte fraction of a human peripheral blood or leukapheresis donation or mobilised apheresis blood collection. Suitably the blood or leukapheresis donation or mobilised apheresis blood donation may be from a healthy subject or a diseased subject.

Suitably the methods may further comprise a step of purifying monocytes from blood, suitably from the mononuclear leukocyte fraction of blood, suitably from the mononuclear leukocyte fraction of a human blood sample. Such purification may comprise isolation of the mononuclear leukocyte fraction, and isolation of purified monocytes from the fraction using specific (markers of monocyte lineage) or non-specific (adherence) methods. Suitably isolation of the mononuclear leukocyte fraction may be carried out by various methods depending upon source material. Suitably isolation of selected purified monocytes may be carried out at small scale (magnetic bead column devices or plastic adherence) or at larger scale for manufacturing using relevant devices such as the CliniMACS Prodigy system (Miltenyi Biotec).

Suitably the monocytes are isolated from whole blood or other cell source as above, suitably by enrichment. Suitably isolation of mononuclear cell fractions may be carried out by density centrifugation or microfluidic separation of the source material. Suitably isolation of purified monocytes may be carried out by filtration of the mononuclear cell fraction, such as for example magnetic bead to a surface marker specific for monocytes with column filtration, suitable filtration systems include the CliniMACS Prodigy system (Miltenyi Biotec). Suitably isolation of purified monocytes may be carried out by CD14 microbead selection.

Suitably the monocytes may be obtained from a peripheral blood or leukapheresis donation from a healthy subject or a diseased subject. Suitably therefore the monocytes may be allogeneic or autologous to the subject. Suitably allogeneic monocytes are obtained from a peripheral blood or leukapheresis donation of a healthy subject. Suitably the healthy subject is blood-group matched to the subject to be treated. Suitably the healthy subject is partially HLA matched to the subject to treat. Suitably the healthy subject is HLA-matched to the subject to be treated.

Suitably, to minimise immune reactions during treatment, the monocytes are derived from a peripheral blood or leukapheresis donation from the diseased subject who is to be treated with the resulting macrophages. In one embodiment, therefore, the monocytes are autologous to subject to be treated. Suitably the monocytes may be obtained from a peripheral blood or leukapheresis donation or mobilised apheresis donation from a diseased subject, the subject having a liver disease or injury. Suitable liver diseases are identified herein below.

Suitably, to ensure supply of monocytes for the methods of the invention, the monocytes are derived from peripheral blood or leukapheresis donation or mobilised apheresis donation from a healthy subject. In one embodiment, therefore, the monocytes are allogeneic to the subject to be treated.

Suitably the monocytes are positive for the expression of the following surface markers—CD14, CD45 and CD192 (CCR2). Suitably the isolated monocytes have low expression of surface marker 25F9 (or the identified molecule recognised by this antibody) and CD206. Suitably the monocytes have high expression of CCR2.

### Macrophages

The present invention relates to novel macrophages produced by a 3-5 day culturing method and macrophages which have particular pro-regenerative properties, optionally produced by the methods herein.

As noted above, suitably the macrophages are monocyte derived macrophages, suitably human monocyte derived macrophages (hMDMs).

Suitably, as noted above, the monocytes used to produce the macrophages of the invention may be autologous to the subject to be treated, therefore suitably the macrophages may be autologous. Suitably therefore the methods of the invention may be methods of producing autologous macrophages, and the macrophages for use in the medical methods of the invention may be autologous macrophages.

Suitably, as noted above, the monocytes used to produce the macrophages of the invention may be allogeneic to the subject to be treated, therefore suitably the macrophages may be allogeneic. Suitably therefore the methods of the invention may be methods of producing allogeneic macrophages, and the macrophages for use in the medical methods of the invention may be allogeneic macrophages.

Suitably, in most embodiments the macrophages are unpolarised. Suitably the macrophages are mature. However, in some embodiments in which a polarisation step is included in the method of production, the macrophages may be polarised, suitably they may be M1-like or M2-like polarised.

In one embodiment, there is provided autologous unpolarised hMDMs produced by the method of the invention. In one embodiment, there is provided autologous unpolarised hMDMs produced by the method of the invention having an anti-inflammatory and anti-fibrogenic phenotype.

In one embodiment, there is provided allogeneic unpolarised hMDMs produced by the method of the invention. In one embodiment, there is provided allogeneic unpolarised hMDMs produced by the method of the invention having an anti-inflammatory and anti-fibrogenic phenotype.

Suitably, the macrophages and any composition comprising the macrophages is GMP-compliant

Suitably, the macrophages produced by the method of the invention are novel by virtue of the method. Suitably in addition, the macrophages produced by the method are in themselves novel by virtue of their cytokine profile. Suitably the macrophages of the invention have a pro-regenerative phenotype. Suitably the macrophages of the invention may have an anti-inflammatory phenotype. Suitably the macrophages of the invention may have an anti-fibrogenic phenotype. Suitably the macrophages may have an anti-inflammatory and anti-fibrogenic phenotype.

Suitably the macrophages may have a cytokine profile which provides the relevant phenotype, or which is indicative of the relevant phenotype. Suitably, the macrophages of the invention have a pro-regenerative cytokine profile. Suitably, the macrophages of the invention are mature macrophages and have a pro-regenerative cytokine profile. Suitably, the macrophages of the invention are mature unpolarised macrophages and have a pro-regenerative cytokine profile.

Suitably by ‘cytokine profile’ it is meant the range of cytokines which are expressed by the macrophages. Typically macrophages may express any of the following cytokines: IL3, IL4, IL6, IL1RA, IL9, TNFa, IL13, IL10, IL17A/F, IL17B, IL17c, and IL17F. The level of expression of each of these cytokines may vary. Suitably the macrophages have a cytokine profile which promotes regeneration and repair in vivo. Suitably the macrophages may have a cytokine profile which would not induce inflammation and/or which would not induce fibrogenesis in vivo. Suitably the macrophages may have a cytokine profile which would reduce inflammation and/or which would reduce fibrogenesis in vivo.

Suitably the macrophages may have increased expression of one or more pro-regenerative cytokines. Suitably the macrophages may have decreased expression of one or more anti-regenerative cytokines.

Suitably the macrophages may have increased expression of one or more anti-inflammatory cytokines, and/or have reduced expression of one or more pro-inflammatory cytokines. Suitably the macrophages may have increased expression of one or more anti-fibrogenic cytokines, and/or have reduced expression of one or more pro-fibrogenic cytokines.

Suitably the macrophages have reduced expression of one or more of the following cytokines: IL1, IL12, IL17 (A, B, C, F), IL18, TNFα, IFNγ, IL4, IL13, PDGF, and TGFβ (1, 2 3). Suitably these cytokines may be regarded as anti-regenerative cytokines.

Suitably the macrophages have reduced expression of one or more of the following inflammatory cytokines: IL1, IL12, IL17 (A, B, C, F), IL18, TNFα, IFNγ. In one embodiment, the macrophages have reduced expression of IL17F.

Suitably the macrophages have reduced expression of one or more of the following fibrogenic cytokines: IL4, IL13, PDGF, TGFβ (1, 2 3). In one embodiment, the macrophages have reduced expression of TGFβ1.

Suitably expression of the anti-regenerative cytokines is reduced in comparison to the level of expression of the same cytokines in macrophages produced by prior art methods. Suitably expression of the inflammatory and fibrogenic cytokines is reduced in comparison to the level of expression of the same cytokines in macrophages produced by prior art methods.

Suitably the macrophages express at least 25% less IL17F as compared to the expression of IL17F by macrophages produced by prior art methods, such as by the 7day method. Suitably the macrophages may express 30% less, 35% less, 40% less, 45% less, 50% less IL17F as compared to the expression of IL17F by macrophages produced by prior art methods, such as by the 7 day method.

Suitably the macrophages express less than 200 pg/mL of IL17F, suitably less than 190 pg/mL of IL17F, suitably less than 180 pg/mL of I L17F, suitably less than 170 pg/mL of IL17F, suitably less than 160 pg/mL of IL17F, suitably 150 pg/mL or less of IL17F.

Suitably the macrophages express at least 2% less TGFβ as compared to the expression of TGFβ by macrophages produced by prior art methods, such as by the 7 day method. Suitably the macrophages may express at least 4% less, 6% less, 8% less, 10% less, 15% less, 20% less TGFβ as compared to the expression of TGFβ by macrophages produced by prior art methods, such as by the 7 day method.

Suitably the macrophages express less than 45000 pg/mL of TGFβ, suitably less than 44000 pg/mL, suitably less than 43000 pg/mL, suitably less than 42000 pg/mL, suitably less than 41000 pg/mL, suitably 40000 pg/mL or less of TGFβ.

Suitably the macrophages express at least 20% less VEGFR1 as compared to the expression of VEGFR1 by macrophages produced by prior art methods, such as by the 7 day method. Suitably the macrophages express at least 20% less, at least 25% less, at least 30% less, at least 35% less, at least 40% less, at least 45% less, at least 50% less VEGFR1 as compared to the expression of VEGFR1 by macrophages produced by prior art methods, such as by the 7 day method.

Suitably the macrophages express more than 50 pg/mL VEGFR1, more than 100 pg/mL of VEGFR1, more than 120 pg/mL of VEGFR1, more than 140 pg/mL VEGFR1, more than 160 pg/mL VEGFR1, more than 170 pg/mL of VEGFR1.

Suitably the macrophages express at least 20% less IL9 as compared to the expression of IL9 by macrophages produced by prior art methods, such as by the 7 day method. Suitably the macrophages express at least 20% less, at least 25% less, at least 30% less, at least 35% less, at least 40% less, at least 45% less, at least 50% less IL9 as compared to the expression of IL9 by macrophages produced by prior art methods, such as by the 7 day method.

Suitably the macrophages express less than 500 pg/mL of IL9, less than 300 pg/mL of IL9, less than 200 pg/mL IL9, less than 180 pg/mL IL9, less than 160 pg/mL IL9, less than 140 pg/mL IL9, less than 130 pg/mL of IL9.

Suitably the level of cytokine expression from macrophages is measured by testing the cell culture supernatant at the end of a given differentiation method using V-plex technology as explained in the examples.

In one embodiment, there is provided an ex vivo generated macrophage having an anti-inflammatory and anti-fibrogenic phenotype, wherein the macrophage expresses less than 200 pg/mL of IL17F and less than 45000 pg/mL of TGFβ.

In one embodiment, there is provided an ex vivo generated macrophage having an anti-inflammatory and anti-fibrogenic phenotype, wherein the macrophage expresses around 150 pg/mL of IL17F and around 40000 pg/mL of TGFβ.

Suitably these cytokine expression levels are averages of the population of macrophages.

Suitably the macrophages of the invention are mature macrophages. Suitably the macrophages express the expected mature macrophage cell surface markers. Suitably the macrophages of the invention are CD14+, CD206+, CD163+, CD169+, 25F9+, and CD86+. Suitably the macrophages are CCR2−.

Suitably the macrophages of the invention have phagocytic capacity. Suitably the macrophages of the invention have the expected phagocytic capacity for mature macrophages. Suitably the macrophages of the invention have a cytoplasmic MFI of between 20-65 after 140 minutes of phagocytosis, of between 30-55 after 140 minutes of phagocytosis, of between 35-50 after 140 minutes of phagocytosis, of about 40 after 140 minutes of phagocytosis. Suitably the cytoplasmic MFI is measured by incubating the macrophages with pH sensitive fluorescent beads for 140 minutes and measuring the emitted fluorescence as explained in the examples.

Suitably the macrophages of the invention respond to inflammatory stimuli. Suitably the macrophages of the invention have the expected response to inflammatory stimuli. Suitably the macrophages of the invention respond to inflammatory stimuli such as: IFNγ, IL10, IL4, IL13, and LPS.

Suitably the macrophages of the invention have low adhesion to surfaces. Suitably the macrophages of the invention have a lower adhesion to surfaces than macrophages produced by prior art methods. Suitably the percentage of adherent macrophages in a population produced by the method is lower than 80%, lower than 75%, lower than 70%, lower than 65%, lower than 60%, lower than 55%, lower than 50%, lower than 45%. Suitably the percentage of adherent cells is calculated by (number of plated cells pre-incubation and washing/number of harvested cells post-incubation and washing)*100. Suitably, these values are calculated as the number of macrophages that are able to adhere to a plastic surface in the span of 2 hours, at 37° C. and 5%CO₂. For example, a 70% adhesion means that 70% of the macrophages seeded onto the plate are attached to it after 2 hours.

### Cell Culture Bag

The macrophages of the invention, a population thereof, or a composition thereof, may be produced i.e. cultured within a cell culture bag.

Suitably, therefore, step (a) of the methods of the invention may take place within a cell culture bag. Suitably therefore monocytes may be differentiated into macrophages in a cell culture bag. Suitably therefore, macrophages may be produced from monocytes by culturing the monocytes in a cell culture bag.

Suitably, the cell culture bag is GMP-graded.

Suitably, the density of monocytes in the cell culture bag is between 1×10⁶/cm² up to 4×10⁶/cm², suitably between 1.5×10⁶/cm² up to 3.5×10⁶/cm², suitably between 1.7×10⁶/cm² up to 3.2×10⁶/cm², suitably between 2×10⁶/cm ² up to 3×10⁶/cm². In one embodiment, the density of monocytes in the cell culture bag is 3×10⁶/cm².

Suitably, the monocytes are cultured in the cell culture bag at the above densities.

### Formulations

The macrophages of the invention are suitably for use in the treatment of any disease in a subject, in particular liver disease or injury in a subject.

Suitably, the macrophages are formulated into medicaments for administration to the subject. Suitably formulation into a medicament may comprise formulation into a composition, suitably a pharmaceutical composition.

Suitably such formulations or compositions are liquids.

Suitably such formulations or compositions may comprise one or more acceptable carriers such as excipients or diluents. Suitably the macrophages are formulated with the one or more acceptable carriers. It will be recognised by one of skill in the art that the form and character of the pharmaceutically acceptable carrier or diluent is dictated by the amount of active ingredient with which it is to be combined, the route of administration and other well-known variables.

Suitably the formulation or composition may comprise pharmaceutically acceptable carriers, including, e.g. , water, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Suitably the formulation or composition may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g. water, alcoholic/aqueous solutions, emulsions or suspensions, including saline and buffered medium.

Suitably pharmaceutically acceptable carriers include, but are not limited to, 0.01-0.1 M and preferably 0.05 M phosphate buffer or 0.8% saline. Other common parenteral carriers include sodium phosphate solutions, Ringer's dextrose, dextrose and sodium chloride, lactated Ringer's, or fixed oils. Intravenous carriers include fluid and nutrient replenishers, electrolyte replenishers, such as those based on Ringer's dextrose, and the like. Preservatives and other additives may also be present such as, for example, antimicrobials, antioxidants, chelating agents, and inert gases and the like.

Suitably, formulations or compositions for injectable use may include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will be preserved against the contaminating action of microorganisms, such as bacteria and fungi. Suitably, the carrier can be a solvent or dispersion medium containing, for example, water, ethanol, polyol (e.g. glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Suitable formulations for use in the therapeutic methods disclosed herein are described in Remington's Pharmaceutical Sciences (Mack Publishing Co.) 16th ed. (1980).

Suitably, prevention of the action of microorganisms can be achieved by sterile manufacturing techniques, various antibacterial and antifungal agents, for example, parabens, chlorobutanol, phenol, ascorbic acid, thimerosal and the like. In many cases, it will be suitable to include isotonic agents, for example, sugars, polyalcohols, such as mannitol, sorbitol, or sodium chloride in the pharmaceutical composition. Prolonged absorption of the injectable compositions can be brought about by including in the formulation or composition an agent which delays absorption, for example, aluminium monostearate and gelatine.

Suitable excipients may include: buffers such as Phosphate Buffered Saline (PBS)/Ethylene Diamine Tetra-Acetic acid (EDTA) buffer (PBS/EDTA buffer) or PBS/EDTA buffer with 0.5% HAS (human albumin serum); saline such as 0.9% saline or 0.9% saline with 0.5% HAS.

Suitably the excipient is 0.9% saline with 0.5% HAS.

Suitably the formulation or composition may further comprise additives, such as antioxidants, or preservatives, for example. Suitably, if the formulation or composition is intended to be frozen, the formulation or composition may further comprise a cryopreservative such as DMSO, suitably 5-10% DMSO.

Suitably the macrophages of the invention may be combined with a second therapeutic agent. Suitably therefore the macrophages of the invention may be formulated into a medicament with a second therapeutic agent.

Suitably the second therapeutic agent may be another agent which is effective to treat a liver disease or injury. Alternatively, second therapeutic agent may be another agent which is effective to treat fibrosis. Suitably the second therapeutic agent to treat a liver disease may be selected from: corticosteroids, interferon, antivirals, bile acids, diuretics, albumin, vitamin K, blood products, and antibiotics, for example. Suitably the second therapeutic agent to treat fibrosis may be selected from: antifibrotic drugs such as those described in (15, 16) or (17).

Suitably the macrophages of the invention may be combined with a second and third therapeutic agent. Suitably the macrophages of the invention may be combined with a second agent effective to treat a liver disease or injury and a third therapeutic agent effective to treat fibrosis.

Suitably the second therapeutic agent may be G-CSF, a CCR2 antagonist, a CCR5 antagonist, or a dual antagonist (18, 19).

### Medical Uses

Suitably the macrophages of the invention are for use as medicaments for treating diseases. Preferred treatment of liver diseases or injuries is described elsewhere herein.

Suitably, as explained above, the macrophages for medical uses are autologous or allogenic.

In one embodiment there is provided autologous macrophages produced by the method of the invention for use as a medicament. In one embodiment, there is provided ex vivo generated autologous macrophages having an anti-inflammatory and anti-fibrogenic phenotype for use as a medicament. Suitably for use in the treatment of a liver disease or injury.

In one embodiment there is provided allogeneic macrophages produced by the method of the invention for use as a medicament. In one embodiment, there is provided ex vivo generated allogeneic macrophages having an anti-inflammatory and anti-fibrogenic phenotype for use as a medicament. Suitably for use in the treatment of a liver disease or injury.

In one embodiment, the macrophages for medical use may be polarised macrophages. In one embodiment, there is provided polarised macrophages produced by the method of the invention for use as a medicament. In one embodiment, there is provided ex vivo generated polarised macrophages having an anti-inflammatory and anti-fibrogenic phenotype for use as a medicament. Suitably for use in the treatment of a liver disease or injury.

Suitably the macrophages may be unpolarised and autologous, or polarised and autologous. Suitably the macrophages may be unpolarised and allogeneic, or polarised and allogeneic.

Suitably any features described herein in relation to medical uses of the macrophages of the invention may equally apply to formulations or pharmaceutical compositions comprising the macrophages of the invention.

Suitably the macrophages of the invention may be provided as a medicament for the treatment of a disease in a subject. Suitably the macrophages of the invention may be formulated so as to be suitable as a medicament for the treatment of a disease in a subject.

Suitable formulations are described elsewhere herein.

Suitably the macrophages are for administration to a subject parenterally, suitably intravenously. Suitably the macrophages are for administration to a subject by injection or infusion. Suitably the macrophages are for administration to a subject intravenously via a peripheral vein.

Suitably the macrophages are for administration to a subject at a dose of about 10⁹ to 10⁹ cells, suitably about 10⁶ to 10⁹ cells, suitably about 10⁷ cells. Suitably an appropriate dose may be determined by the medical professional based on weight, sex and age of the subject for example.

Suitably the macrophages are for administration to a subject in a single dose or multiple doses. Suitably doses may be given at intervals. Suitably doses may be given three times a day, once a day, once every two days, once every four days, once a week, once every two weeks, once every month, once every two months, once every few months, once a year, for example.

In one embodiment, the macrophages are for administration to a subject at an interval of once per month. In one embodiment, the macrophages are for administration to a subject in three doses at an interval of once per month. In one embodiment, the macrophages are for administration to a subject in three doses of 10⁹ cells at an interval of once per month.

Suitably the macrophages of the invention are for use as a medicament.

Suitably the macrophages of the invention are for use in the treatment of a disease or injury.

Suitably the macrophages of the invention are for use in the treatment of an acute or chronic disease or injury. Suitably the macrophages of the invention are for use in the treatment of an acute injury. Suitably the macrophages of the invention are for use in the treatment of a chronic disease.

Suitably the macrophages of the invention are for use in the treatment of an acute or chronic disease or injury by improving regeneration, suitably by reducing expression of anti-regenerative cytokines.

Suitably the macrophages of the invention are for use in the treatment of an acute or chronic disease or injury by reducing fibrosis and/or reducing inflammation, suitably by reducing expression of pro-fibrotic and/or pro-inflammatory cytokines.

Suitably therefore macrophages may be for use in the treatment of fibrotic or inflammatory diseases, suitably for use in the treatment of diseases which involve fibrosis and/or inflammation. Suitably the transfected macrophages may be for use in the treatment of a disease by reducing fibrosis and/or by reducing inflammation.

Suitably the macrophages may be for use in the treatment of any inflammatory disease, or any fibrotic disease.

Suitably the macrophages may be for use in the treatment of any liver disease, kidney disease, lung disease, or muscle disease. Suitably the macrophages may be for use in the treatment of any fibrotic disease or inflammatory disease in the liver, kidney, lung, or muscle. Suitably the transfected macrophages may be for use in the treatment of any fibrotic disease or inflammatory disease in the liver.

Suitably the macrophages may be for use in the treatment of fibrotic liver disease, fibrotic kidney disease, fibrotic lung disease, or fibrotic muscle disease. Suitably the macrophages may be for use in the treatment of an inflammatory liver disease, inflammatory kidney disease, inflammatory lung disease, or inflammatory muscle disease. Suitably the macrophages may be for use in the treatment of liver diseases, kidney diseases, lung diseases, or muscle diseases by reducing fibrosis and/or inflammation. Suitably the macrophages may be for use in the treatment of fibrotic liver diseases or inflammatory liver diseases. Suitably the macrophages may be for use in the treatment of liver diseases by reducing fibrosis and/or reducing inflammation.

Suitable liver diseases include: chronic liver disease, acute liver disease, acute-on-chronic liver disease, Alagille Syndrome, Alcohol-Related Liver Disease, acute fatty liver of pregnancy, Alpha-1 Antitrypsin Deficiency, Autoimmune Hepatitis, Benign Liver Tumours, Biliary Atresia, Budd Chiari syndrome, Cirrhosis, Crigler-Najjar Syndrome, Cystic fibrosis related liver disease, Gallstones, Galactosemia, Gilbert Syndrome, Hemochromatosis, Hepatic Encephalopathy, Hepatitis A, Hepatitis B, Hepatitis C, Hepatitis E, Hepatorenal Syndrome, Intrahepatic Cholestasis of Pregnancy (ICP), Lysosomal Acid Lipase Deficiency (LAL-D), Liver Cysts, Liver abscesses, Liver Cancer, Newborn Jaundice, Non-Alcoholic Fatty Liver Disease, Non-Alcoholic Steatohepatitis, Primary Biliary Cholangitis (PBC), Porphyria, Portal hypertension, Primary Sclerosing Cholangitis (PSC), Progressive Familial Intrahepatic Cholestasis (PFIC), Reye Syndrome, Type I Glycogen Storage Disease, Wilson Disease.

Suitably lung diseases include: chronic lung disease, acute lung disease, acute-on-chronic lung disease, asthma, COPD, pneumonia, emphysema, pulmonary fibrosis, lung cancer, mesothelioma, cystic fibrosis, tuberculosis, respiratory infections, pulmonary edema, bronchitis, pulmonary embolism, pulmonary hypertension, sarcoidosis, interstitial lung disease, Langerhans cell histiocytosis, bronchiolitis obliterans, post inflammatory pulmonary fibrosis, pulmonary alveolar proteinosis, idiopathic pulmonary hemosiderosis, pulmonary alveolar microlithiasis, idiopathic interstitial pneumonia, idiopathic pulmonary fibrosis, acute interstitial pneumonitis, cryptogenic organising pneumonia, desquamative interstitial pneumonia, lymphangioleiomyomatosis, neuroendocrine cell hyperplasia, pulmonary interstitial glycogenosis, alveolar dysplasia, rheumatoid lung disease, cytokine release syndrome (CRS)—induced acute respiratory distress syndrome (ARDS) and secondary hemophagocytic lymphohistiocytosis (sHLH).

Suitable kidney diseases include: chronic kidney disease, acute kidney disease, acute-on-chronic kidney disease, Abderhalden-Kaufmann-Lignac syndrome (Nephropathic Cystinosis), Acute Kidney Failure/Acute Kidney Injury, Acute Lobar Nephronia, Acute Phosphate Nephropathy, Acute Tubular Necrosis, Adenine Phosphoribosyltransferase Deficiency, Apparent Mineralocorticoid Excess Syndrome , Arteriovenous Malformations and Fistulas of the Urologic Tract, Autosomal Dominant Hypocalcemia, Bardet-Biedl Syndrome, Bartter Syndrome, Beer Potomania, Beeturia, β-Thalassemia Renal Disease, Bile Cast Nephropathy, Birt-Hogg-Dubé Syndrome, C1q Nephropathy, C3 Glomerulopathy C3 Glomerulopathy with Monoclonal Gammopathy, C4 Glomerulopathy, CAKUT (Congenital Anomalies of the Kidney and Urologic Tract), Capillary Leak Syndrome, Cardiorenal syndrome, CFHR5 nephropathy, Charcot-Marie-Tooth Disease with Glomerulopathy, Churg-Strauss syndrome, Chyluria, Ciliopathy, Cold Diuresis, Collagenofibrotic Glomerulopathy, Collapsing Glomerulopathy, Congenital Anomalies of the Kidney and Urinary Tract (CAKUT), Congenital Nephrotic Syndrome, Congestive Renal Failure, Conorenal syndrome (Mainzer-Saldino Syndrome or Saldino-Mainzer Disease), Contrast Nephropathy, Cortical Necrosis, Cryocrystalglobulinemia, Cryoglobuinemia, Crystal-Storing Histiocytosis, Cystinuria, Dense Deposit Disease (MPGN Type 2), Dent Disease (X-linked Recessive Nephrolithiasis), Dialysis Disequilibrium Syndrome, Diabetes and Diabetic Kidney Disease, Diuresis, Drug and substance induced kidney disease, EAST syndrome, Ectopic Kidney, Erdheim-Chester Disease, Fabry's Disease, Familial Hypocalciuric Hypercalcemia, Fanconi Syndrome, Fraser syndrome, Fibrillary Glomerulonephritis and Immunotactoid Glomerulopathy, Fraley syndrome, Hypervolemia, Focal Segmental Glomerulosclerosis, Focal Glomerulosclerosis, Galloway Mowat syndrome, Hypertension, Gitelman Syndrome, Glomerular Diseases, Glomerular Tubular Reflux, Glycosuria, Goodpasture Syndrome, HANAC Syndrome, Heat Stress Nephropathy, Hemolytic Uremic Syndrome (HUS), Atypical Hemolytic Uremic Syndrome (aHUS), Hemophagocytic Syndrome, Hemorrhagic Cystitis, Nephropathis Epidemica), Hemosiderinuria, Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatocyte Nuclear Factor 1β-Associated Kidney Disease, Hepatorenal Syndrome, HNF1B-related Autosomal Dominant Tubulointerstitial Kidney Disease, Horseshoe Kidney (Renal Fusion), Hunner's Ulcer, Hydrophilic Polymer Emboli, Hyperaldosteronism, Hypercalcemia, Hyperkalemia, Hypermagnesemia, Hypernatremia, Hyperoxaluria, Hyperphosphatemia, Hypocalcemia, Hypocomplementemic Urticarial Vasculitic Syndrome, Hypokalemia-induced renal dysfunction, Hypomagnesemia, Hyponatremia, Hypophosphatemia, Interstitial Nephritis, Infection induced kidney disease, Ivemark's syndrome, Joubert Syndrome, Kidney Stones, Nephrolithiasis, Kidney cancer, Lecithin Cholesterol Acyltransferase Deficiency (LCAT Deficiency), Liddle Syndrome, Lightwood-Albright Syndrome, Lipoprotein Glomerulopathy, Lupus, Systemic Lupus Erythematosis, Lysinuric Protein Intolerance, Lysozyme Nephropathy, Malignancy-Associated Renal Disease, Malakoplakia, McKittrick-Wheelock Syndrome, Meatal Stenosis, Medullary Cystic Kidney Disease, Urolodulin-Associated Nephropathy, Juvenile Hyperuricemic Nephropathy Type 1, Medullary Sponge Kidney, MELAS Syndrome, Membranoproliferative Glomerulonephritis, Membranous Nephropathy, MesoAmerican Nephropathy, Metabolic Acidosis, Metabolic Alkalosis, Microscopic Polyangiitis, Milk-alkalai syndrome, Dysproteinemia, MUC1 Nephropathy, Multicystic dysplastic kidney, Multiple Myeloma, Myeloproliferative Neoplasms and Glomerulopathy, Nail-patella Syndrome, NARP Syndrome, Nephrocalcinosis, Nephrocystin-1 Gene Deletions and ESRD, Nephrogenic Systemic Fibrosis, Nephronophthisis due to Nephrocystin-1 Gene Deletions, Nephroptosis (Floating Kidney, Renal Ptosis), Nephrotic Syndrome, Nodular Glomerulosclerosis, Nutcracker syndrome, Oligomeganephronia, Orotic Aciduria, Oxalate Nephropathy, Page Kidney, Papillary Necrosis, Papillorenal Syndrome (Renal-Coloboma Syndrome, Isolated Renal Hypoplasia), The Peritoneal-Renal Syndrome, POEMS Syndrome, Podocyte Infolding Glomerulopathy, Post-infectious Glomerulonephritis, Polyarteritis Nodosa, Polycystic Kidney Disease, Posterior Urethral Valves, Post-Obstructive Diuresis, Proliferative Glomerulonephritis with Monoclonal IgG Deposits (Nasr Disease), Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Stenosis, Renal Cell Cancer, Renal Cyst, Renal Infarction, Renal Osteodystrophy, Renal Tubular Acidosis, Retroperitoneal Fibrosis, Rhabdomyolysis, Rheumatoid Arthritis-Associated Renal Disease, Sarcoidosis Renal Disease, Salt Wasting, Scleroderma Renal Crisis, Serpentine Fibula-Polycystic Kidney Syndrome, Exner Syndrome, Sickle Cell Nephropathy, TAFRO Syndrome, Tea and Toast Hyponatremia, Thin Basement Membrane Disease, Benign Familial Hematuria, Thrombotic Microangiopathy Associated with Monoclonal Gammopathy, Trench Nephritis, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumour Lysis Syndrome, Uremia, Uremic Optic Neuropathy, Ureteritis Cystica, Ureterocele, Urethral Caruncle, Urethral Stricture, Urinary Tract Infection, Urogenital Fistula, Uromodulin-Associated Kidney Disease, Vasomotor Nephropathy, Vesicointestinal Fistula, Vesicoureteral Reflux, VGEF Inhibition and Renal Thrombotic Microangiopathy, viral induced kidney disease, Von Hippel-Lindau Disease, Waldenstrom's Macroglobulinemic Glomerulonephritis, Wegener's Granulomatosis, Granulomatosis with Polyangiitis, Wunderlich syndrome, Zellweger Syndrome, Cerebrohepatorenal Syndrome.

Suitable muscle diseases include: chronic muscle diseases, acute muscle diseases, acute-on-chronic muscle diseases, muscular dystrophies (e.g. Duchenne muscular dystrophy, limb girdle muscular dystrophies), idiopathic inflammatory myopathies (e.g. Dermatomyositis, Polymyositis), Myasthenia gravis, Amyotrophic Lateral Syndrome, Mitochondrial myopathies, Rhabdomyolysis, Fibromyalgia, sprains and strains, and Muscle tumours, such as leiomyomas, rhabdomyomas, and rhabdomyosarcomas.

### Liver Disease or Injury

The macrophages of the invention are preferably for use in the treatment of liver disease or injury.

Suitably the macrophages of the invention may be for use in the treatment of cirrhosis in a liver disease or injury. Suitably the macrophages of the invention may be for use in the treatment of inflammation and/or fibrosis in a liver disease or injury.

Suitably the macrophages of the invention may be for use in the treatment of liver disease or injury by reducing fibrosis, by reducing necrosis, by increasing liver cell proliferation, by reducing bilrubinemia, by reducing GLDH, by increasing liver:weight ratio, by reducing AST levels; and/or by reducing inflammation (full detail of liver function test interpretation provided in Materials and Methods).

Suitably the liver disease or injury may be chronic, or acute, or acute-on-chronic. Suitably an acute liver disease or injury may be classed as a liver disease or injury with an onset of less than 24 weeks from cause. Suitably a chronic liver disease may be classed as a liver disease or injury which has persisted for more than 6 months. Suitably an acute-on chronic liver disease may be classed as a liver disease or injury with an onset of less than 24 weeks from cause in a patient that already has a chronic liver disease that has persisted for more than 6 months.

Suitably a chronic liver disease/injury may be selected from the following: hepatitis C; hepatitis B; alcohol related liver disease; non-alcoholic fatty liver disease; cryptogenic cirrhosis; Wilson's disease; autoimmune hepatitis; cholangitis; hemochromatosis; and alpha-1-antitrypsin deficiency.

Suitably the macrophages are for use in the treatment of liver disease or injury by reducing fibrosis and/or inflammation in a chronic liver disease. Suitably the macrophages are for use in the in the treatment of chronic liver disease by reducing fibrosis and/or inflammation.

Suitably an acute liver disease/injury may be caused by the following: excessive alcohol consumption; adverse reaction to medications; poisoning for example by food, chemicals, toxins; infection with microorganisms such as cytomegalovirus, Epstein Barr virus, yellow fever; acute fatty liver of pregnancy; and drug overdose, for example acetaminophen overdose (APAP).

Suitably the macrophages are for use in the treatment of liver disease or injury by reducing necrosis in an acute liver disease. Suitably the macrophages are for use in the in the treatment of acute liver disease by reducing necrosis.

Suitably the macrophages for treatment of acute liver disease/injury are polarised.

Suitably therefore polarised macrophages of the invention may be for use in the treatment of liver disease or injury by reducing necrosis in acute liver disease. Suitably the polarised macrophages of the invention are for use in the treatment of acute liver disease by reducing necrosis.

Suitably the macrophages for treatment of chronic liver disease/injury are non-polarised.

Suitably therefore non-polarised macrophages of the invention are for use in the treatment of liver disease or injury by reducing fibrosis in a chronic liver disease. Suitably the non-polarised macrophages of the invention are for use in the in the treatment of chronic liver disease by reducing fibrosis.

In one embodiment, the macrophages of the invention are for use in the treatment of liver cirrhosis.

In one embodiment, the macrophages of the invention are for use in the treatment of APAP overdose.

Suitably, the macrophages for use in the treatment of liver cirrhosis are non-polarised macrophages.

Suitably, the macrophages for use in the treatment of a drug overdose are polarised macrophages. Suitably the macrophages for use in the treatment of a drug overdose are M2 macrophages.

In one embodiment, there is provided M2 polarised macrophages produced by the method of the invention for use in the treatment of an APAP overdose.

### Subject

The macrophages of the invention are for use as a medicament for treating a disease in a subject in need thereof.

Suitably the subject may be a human or animal, suitably the subject is a human. Suitably the subject may be a child or an adult, suitably the subject is an adult.

Suitably the subject may be in need of treatment. Suitably therefore the subject may have a disease, or be at risk of developing a disease. Suitably the subject may have, or be at risk of developing, a liver disease or injury as defined hereinabove.

Suitably the subject may satisfy certain risk factors associated with liver disease or injury, for example: alcoholism, drug abuse, obesity, an autoimmune disorder, metabolic syndrome, taking certain medications, exposure to toxic chemicals/microorganisms.

Suitably the subject may have symptoms of a disease. Suitably the subject may have symptoms associated with a liver disease or injury.

Suitably the subject may have one or more of the following symptoms: nail clubbing, palmar erythema, angiomata, gynaecomastia, testicular atrophy, anaemia, caput medusae, drowsiness, hyperventilation, asterixis, jaundice, ascites, leukonychia, peripheral oedema, bruising, respiratory alkalosis, liver enlargement, dupuytren's contracture, parotid enlargement, peripheral neuropathy, and kayser-fleisher rings, for example.

Suitably the subject may have a MELD score which ranks the severity of liver disease or injury of the subject. Suitably the subject may have a MELD score which is between 10-16.

Suitably the subject may have a MELD score which ranks the severity of liver disease or injury of the subject. Suitably the subject may have a MELD score which is less than 20, optionally the subject may have a MELD score between 10-19.

### FIGURES

FIG. 1: shows characterisation of the inventive protocol (day5 no feed) vs. standard protocol (day 7 plus re-feed) A Representative pictures of day5 no feed macrophages (day5) and day7 plus feed macrophages (day7) from two distinct donors (brightfield, 20×, scale bar=200 mm); B Yield of the day5 no feed vs. the day7 plus feed protocol. The percentage (%) yield is calculated as: (Number of macrophages at harvest/number of macrophages plated)*100. D'Agostino and Pearson omnibus normality test was carried out, and then a non-parametric Mann Whitney test was applied. p=0.0641; C Viability at the end of the day5 no feed vs. the day7 plus feed protocol. The percentage (%) is calculated as: % of DRAQ-CD45+ macrophages in the single cell gate. D'Agostino and Pearson omnibus normality test was carried out, and then a non-parametric Mann Whitney test was applied. p=0.99; D-E Gas chromatography results on supernatants of five day5 no feed and five day7 plus feed cell culture supernatants. Glucose (D) and lactate (E) are measured. Sample too small to carry out a D'Agostino and Pearson omnibus normality test: a non-parametric Mann Whitney test was applied. p=0.99; F Adhesion properties of the day5 no feed and day7 plus feed macrophages. The percentage (%) of adhesive cells is calculated as: (number of plated cells pre-incubation and washing/number of harvested cells post-incubation and washing)*100. Sample too small to carry out a D'Agostino and Pearson omnibus normality test: a non-parametric Wilcoxon signed rank test was applied. p=0.1875.

FIG. 2: shows further characterisation of the inventive protocol (day5 no feed) vs. standard protocol (day 7 plus re-feed) A Dosage of functionally relevant cytokines using the V-Plex technology. Data are expressed in pg/mL. Day5 and day7 concentrations are calculated as reported in materials and methods. The same donors are tested for day5 and day7 cytokine secretion. Data are analysed using t-test for paired data. **p<0.01; ***p<0.001; B Flow cytometry analysis of cell surface markers. Each dot represents a donor. MFI ratio is calculated dividing the day5 or day7 MFI by the MFI of CD14+ monocytes at the start of the culture. The dotted line represents a ratio of 5, considered the minimal target ratio for CD206 and 25F9 to release the day7 macrophages for treatment. Data are analysed using t-test for unpaired data; C Phagocytosis assay using live imaging. We report the mean±SD of the macrophages' cytoplasmic MFI in the AlexaFluor488 channel (i.e. of the ingested beads). There are no significant differences at any of the data point analysed. Data are analysed using a two-way ANOVA.

FIG. 3: shows further characterisation of the inventive protocol (day5 no feed) vs. standard protocol (day 7 plus re-feed) A Yield of day5 and day7 protocols with and without feed. Each symbol is an independent donor; B Yield of day5 and day7 protocols with and without feed, with and without human serum type AB (hserum AB); C Gas chromatography analysis of Glucose in the cell culture supernatants of hMDMs cultured in AimV (AV) or TexMacs (TM) with or without feed, with or without serum. Each symbol is an independent donor; D Gas chromatography analysis of Lactate in the cell culture supernatants of hMDMs cultured in AimV (AV) or TexMacs (TM) with or without feed, with or without serum. Each symbol is an independent donor; E Viability of cells using day5 protocols with and without feed, with and without human serum type AB (hserum AB) F Day-by-day analysis of viability by flow cytometry in two donors using a no-feed and a feed protocol. All data are reported as mean±SD.

FIG. 4: shows characterisation of the day-by-day differentiation of monocytes into macrophages, with or without feeding, A-H Flow cytometry analysis of cell surface markers. At each time point, each symbol represents monocyte-macrophages from a single donor over time. Monocyte-macrophages with and without feed are represented with different symbols (). MFI ratio is calculated dividing the dayₓ MFI by the MFI of CD14+ monocytes at the start of the culture. The specific marker analysed is reported in the label of the Y axis.

FIG. 5: shows characterisation of the inventive protocol (day5 no feed) vs. standard protocol (day 7 plus re-feed) A Dosage of functionally relevant cytokines using the V-Plex technology. Data are expressed in pg/mL. Data from day5 and day7 polarised macrophages are clusterised using the online platform Morpheus. The same donors are tested for day5 and day7 cytokine secretion. The false-colour scale ranges from light grey to dark grey to represent minimal to maximal expression of any given protein. Grey box represents samples that were below the detection limit of the assay; B-D Representative cytokines and chemokines are reported (IL17A/F, MCP-1 and IL10 respectively). Each symbol is a distinct donor. Data are shown as mean±SD. Dots of the same shape represents samples polarised with the same cue; E-H Flow cytometry analysis of cell surface markers. Each symbol represents a donor. MFI ratio is calculated dividing the MFI of day5 or day7 polarised hMDMs by the MFI of day5 or day7 unpolarised hMDMs. The dotted line represents a ratio of 1: a ratio above 1 means upregulation in polarised vs unpolarised hMDMs. A ratio below 1 means downregulation.

FIG. 6: shows characterisation of various differentiation and polarisation protocols A Flow cytometry analysis of cell surface markers of hMDMs D5 and D7 polarised using a combination of rIL4 and rIL13 (20 ng/mL). Each symbol is an independent donor. Data are reported as RFI, calculated as MFI polarised/MFI unpolarised. The dotted line set at 1 on the graph represents therefore no modulation of the specific marker as compared to the unpolarised cells; B Flow cytometry analysis of CD206 in day5 hMDMs cultured in GMP cell culture bags at distinct densities. RFI is calculated dividing the MFI of the day5 hMDMs by the MFI of CD14+monocytes. Lines connect RFI values measured in hMDMs from a single donor; C Flow cytometry analysis of 25F9 in day5 hMDMs cultured in GMP cell culture bags at distinct densities. RFI is calculated dividing the MFI of the day5 hMDMs by the MFI of CD14+ monocytes. Lines connect RFI values measured in hMDMs from a single donor.

FIG. 7: shows characterisation of the day5 no feed protocol in GMP bags vs. standard plastic A Yield of the day5 no feed protocol in GMP bags using three distinct cell densities: 10⁶/cm², 2×10⁶/cm² and 3×10⁶/cm². Each line connects data from a single donor, according to legend; B Viability of the hMDMs produced with the day5 no feed protocol in GMP bags using three distinct cell densities: 10⁶/cm², 2×10⁶/cm² and 3×10⁶/cm². Each line connects data from a single donor, according to legend; C Yield of the day5 no feed protocol in GMP bags vs. standard plastic. The percentage (%) yield is calculated as: (Number of macrophages at harvest/number of macrophages plated)*100. D'Agostino and Pearson omnibus normality test was carried out, and then a non-parametric Mann Whitney test was applied; D Viability at the end of the day5 no feed protocol in GMP bags vs. standard plastic. The percentage (%) is calculated as: % of DRAQ-CD45+ macrophages in the single cell gate. D'Agostino and Pearson omnibus normality test was carried out, and then a non-parametric Mann Whitney test was applied; E Flow cytometry analysis of cell surface markers. Each symbol represents a donor. MFI ratio is calculated dividing the day5 MFI by the MFI of CD14+ monocytes at the start of the culture. The dotted line represents a ratio of 4, considered the minimal target ratio for CD206 and 25F9 to release the day7 macrophages for treatment. Data are analysed using t-test for unpaired data.

FIG. 8: shows injection of day5 hMDMs in mouse models of chronic and acute liver injury A Experimental design: NOD/SCID mice are injected twice a week for 12 weeks with CCl₄to induce severe liver fibrosis. Day5 hMDMs are injected at the start of week 9, 10 and 11. Mice are culled one week later the last dose of macrophage therapy; B PSR staining is performed on the livers treated with day5 hMDMs or saline and stained areas are quantified. Each symbol represents a mouse. 6 to 10 10× fields/mouse are quantified. D'Agostino and Pearson omnibus normality test was carried out, and then a non-parametric Mann Whitney test was applied. p=0.12; C-D Dosage of circulating serum ALT (C) and bilirubin (D) at cull. Each dot represents a mouse. D'Agostino and Pearson omnibus normality test was carried out, followed by a one-tailed t-test for unpaired data with Welch's correction for samples with unequal variances. p non-significant for serum ALT, although a trend towards reduction in day5 hMDMs treated mice is reported (p=0.07); serum bilirubin was significantly reduced in day5 hMDMs treated mice at cull: *p<0.05 (p=0.02) for bilirubin; E Representative pictures of day5 hMDMs and saline treated livers. (10×, brightfield); F Experimental design: C57Bl/6 WT mice were starved for 14 h prior the injection of paracetamol (acetaminophen, APAP). Day5 hMDMs polarised to AAMs are injected 16 h post-APAP injection and mice culled 2 0h later (36 h post-AAP injection); G H&E staining is performed on the livers treated with day5 AAMs or saline and stained areas are quantified. Each dot represents a mouse. 6 to 10 10× fields/mouse are quantified. A Shapiro-Wilk normality test was applied, followed by a t-test for unpaired data was applied. p=0.13; H-I Dosage of circulating serum GLDH and AST. Each dot represents a mouse. A Shapiro-Wilk normality test was carried out, followed by a one-tailed t-test for unpaired data with Welch's correction for samples with unequal variances. For GLDH *p<0.05 (p=0.046), for AST p non-significant (p=0.263). J Representative haematoxylin and eosin stainings of livers treated with vehicles or day5 AAMs.

FIG. 9: shows the supporting data for the in vivo experiments carried out in FIG. 8. A Weight was monitored twice a week for 12 weeks during CCl₄ treatment. Data mouse by mouse are plotted. We report in the vehicle treated mice and the hMDMs day5 treated mice. B Liver:weight ratio is not significantly different in vehicle vs. day5 hMDMs treated mice at the point of cull. C-E Serum levels of ALP (C), bilirubin (D) and albumin (E) in vehicle vs. day5 hMDMs treated mice at cull. F-H White Blood Cells (WBC, F), Red Blood Cells (RBC, G) and haematocrit (HCT, H) in vehicle vs. day5 hMDMs treated mice at cull. We reported a trend towards reduction in RBC and HCT in day5 hMDMs as compared to vehicle treated mice. I-J Serum IL6 (I) and IL10 (J) in vehicle vs. day5 hMDMs treated mice at cull. No difference are reported. A-J A Shapiro-Wilk normality test was carried out, followed by a one-tailed t-test for unpaired data with Welch's correction for samples with unequal variances. p<0.05 was considered statistically significant. K Liver:body weight ratio at cull 36 h after paracetamol (APAP) overdose in vehicle vs. day5 hMDMs treated mice at cull. L Weight drop at cull in vehicle vs. day5 hMDMs treated mice at cull. M-O Serum levels of ALP (M), bilirubin (N) and albumin (0) in vehicle vs. day5 hMDMs treated mice at cull. Bilirubin was significantly lower in day5 hMDMs treated as compared to vehicle treated mice. P-R White Blood Cells (WBC, P), Red Blood Cells (RBC, Q) and haematocrit (HCT, R) in vehicle vs. day5 hMDMs treated mice at cull. K-R A one-tailed t-test for unpaired data with Welch's correction for samples with unequal variances was carried out. p<0.05 was considered statistically significant. B-R Every dot represents a distinct mouse.

### EXAMPLES

The present invention is further exemplified by the following examples. The examples are for illustrative purpose only and are not intended, nor should they be construed as limiting the invention in any manner.

Certain embodiments of the invention will now be demonstrated by way of the following non-limiting examples and with reference to the figures above.

### Materials and Methods

### GMP Human Monocyte-Derived Macrophages (hMDMs) Cell Culture

We isolated monocytes from a buffy coat product from a healthy volunteer sourced from the Scottish National Blood Transfusion Service (SNBTS) using a Ficoll gradient (GE Healthcare) followed by a magnetic column selection using CliniMACS CD14 Reagent (Miltenyi Biotec). We then matured monocytes for 1 to 7 days in culture in TexMACS without phenol red (Miltenyi Biotec) in the presence of 100 ng/mL GMP-graded recombinant human macrophage colony-stimulating factor (rhM-CSF) (R&D System, Biotechne). hMDMs day5 and day7 are cultured in 6 wells multi-well plate (Corning Costar) at a density of 2×10⁶/cm². hMDMs d5 are also cultured in GMP cell culture bags (Miltenyi Biotec) using increasing concentrations: 1×10⁶/cm², 2×10⁶/cm² and 3×10⁶/cm². hMDMs were fed at day 3 when matured for 7 days: briefly, half of the culture medium volume is added to each well/bag, supplemented with rhM-CSF at a final concentration of 100 ng/mL. Day5 and day7 hMDMs were counted using an automated counter (TC20, BioRad).

### Flow Cytometry

hMDMs were harvested and spun at 300× g, 5 minutes, room temperature. hMDMs were re-suspended at a concentration of 10⁶/mL in PBS +2.5 mM EDTA+0.5% Albumin (PEA). Blocking was performed by incubating hMDMs in PEA with FcR blocking reagent (Miltenyi Biotec) 1:100 for 20 minutes at 4° C. Antibodies were added to the cell suspension at a dilution of 1:100 and incubated for 20 minutes at 4° C. (details of the antibodies used are reported in the table below).

TABLE 1
Antibodies

| ANTIGEN | FLUOROPHORE | CLONE | SUPPLIER | ORDERING CODE |
|---|---|---|---|---|
| CD45 | VB | 5B1 | Miltenyi Biotec | 130-092-880 |
| CD14 | VB/PE | TUK4 | Miltenyi Biotec | 130-091-242/130-113-152 |
| CD206 | FITC | | Miltenyi Biotec | 130-095-131 |
| 25F9 | APC | 25F9 | eBioscience | 50-0115-42 |
| CCR2 | PE | K036C2 | BioLegend | 357206 |
| CD163 | FITC | | Miltenyi Biotec | BO-097-626 |
| CD169 | APC | 7-239 | BioLegend | 346008 |
| CD86 | PE | BU63 | BioLegend | 374206 |
| MHC II | FITC | TU39 | BioLegend | 361705 |

Data were acquired using the Miltenyi Vyb flow cytometer and analysed with the MACS Quant software (Miltenyi Biotec).

### V-Plex Cytokine Dosage

Cytokines in cell culture supernatants: cytokines were analysed using a V-PLEX Human Biomarker 54-Plex kit on a MESO Quickplex SQ 120 according to the manufacturers' instructions (Meso Scale Discovery). TGF-β1, TGF-β32 and TGF-β33 were analysed using the TGF-b V-plex kit on a MESO Quickplex SQ 120 according to the manufacturers' instructions (Meso Scale Discovery). Cytokines belonging to the IL17 family (IL17A/F, IL17B, IL17C, IL17F) were analysed using a V-plex kit on a MESO Quickplex SQ 120 according to the manufacturers' instructions (Meso Scale Discovery). 10 μL of supernatants were tested. Results are in pg/mL. Values are adjusted taking into consideration the distinct time in culture (hMDMs day7 secrete cytokines for 2 days more), higher dilution of cytokines in hMDMs day7 (they receive ⅓ more of medium as a result of the feed at day3) and average yield (yield of hMDMs d5 is higher than hMDMs d7).

### Mouse Experiments

NOD CB17 Prkdc/^{SCID} mice were supplied by Charles River and housed in individually ventilated cages in a sterile animal facility with a 10-14-hours dark/light cycle and free access to food and water. All procedures were performed in accordance with UK Home Office guidelines (Animals [Scientific Procedures] Act 1986). Chronic liver fibrosis was induced in adult male mice over a 12-week period by twice weekly intraperitoneal injections of carbon tetrachloride (CCl₄) dissolved in sterile olive oil at a concentration of 0.2 mL/kg for the first week increasing to 0.4 mL/kg for the remaining 11 weeks. One day after the 18th CCl₄ injection (9 weeks), mice were randomly allocated to receive either day5 hMDMs (n=10) or saline (vehicle, n=9) injections via tail vein. The intra-splenic route would have ensured maximal cell delivery, but it does not model the administration route used in the phase I MATCH trial (day7 hMDMs in patients with chronic liver fibrosis) (14). Day 5 hMDMs were suspended in sterile saline at a density of 5×10⁷ cells/mL and 0.1 mL was injected via a 30-gauge needle (Myjector 0.3 mL syringes, Terumo). Day5 hMDMs intravenous injection was repeated at week 10 and week 11. CCl₄ administration continued for an additional week.

Wild-type C57BL/6J male mice (8-10 weeks old) were allowed to acclimatise for a minimum of one week in a clean animal facility. Prior to paracetamol (APAP) administration, mice were fasted at least 12 hours. Mice received a single injection (i.p.) of APAP (350 mg/kg) dissolved in warm saline between 20:00 and 22:00. Mice were left recovery until morning in a heated cabinet (27° C.); 16 h post-APAP overdose, they received hMDMs day5 polarised towards alternatively activated phenotype (AAMs) using rhIL4+rhIL13 (20 ng/mL) and rhM-CSF (100 ng/mL) for 24 h.

All mice were culled at the indicated time points using anaesthesia overdose followed by cervical dislocation as confirmatory method. Organs and blood were retrieved, processed and stored for further analysis: liver left lobe was snap frozen and stored at −80° C.; the other liver lobes were fixed in formalin 10% for 8 h and then included in paraffin blocks; kidneys, spleen, heart and lungs were fixed in formalin 10% for 8 h and then included in paraffin blocks; blood was collected in Eppendorf, left to sediment for 8 h and then spun at 10000× g for 10 minutes at room temperature to obtain serum, to be stored at −80° C.; blood collected in EDTA-coated tubes (Microvette CB300, Sarstedt) were used to collect 30 μL of full blood to use for the analysis of the haematological parameters using the CellTac machine (Nihon Kohden).

### Liver Function Tests on Sera

Serum chemistry was performed by measurement of alanine aminotransferase (ALT), aspartate aminotransferase (AST), alkaline phosphatase (ALP), total bilirubin, and serum albumin. ALT was measured using a commercial kit (Alpha Laboratories Ltd). AST and ALP were determined by a commercial kit (Randox Laboratories). Total bilirubin was determined by the acid diazo method described by Pearlman and Lee (20) using a commercial kit (Alpha Laboratories Ltd). Mouse serum albumin measurements were determined using a commercial serum albumin kit (Alpha Laboratories Ltd). All kits were adapted for use on a Cobas Fara centrifugal analyzer (Roche Diagnostics Ltd). For all assays, intra-run precision was CV<4%. In some experiments, assays were run on plasma samples with the exception of ALP activity.

Here below it is reported a table to highlight which parameters are more helpful to evaluate liver damage in acute and chronic models (+++=extremely helpful; ++=very helpful; +=helpful; ±=moderately helpful; −=not helpful).

TABLE 2
Liver Damage Parameters

| | ALT | AST | GLDH | ALP | BILIRUBIN |
|---|---|---|---|---|---|
| Chronic CCl₄ | + | + | − | ± | +++ |
| Acute APAP O.D. | ++ | ++ | +++ | ± | ++ |

### Histological Analysis

Haematoxylin and eosin (H&E) and picrosirius red (PSR) staining were performed according to standard protocols. Morphometric pixel analysis to quantify histological staining was performed. For necrosis and fibrosis quantification respectively, H&E and PSR stained section were scanned to create a single image with Polaris slide scanner (Perkin Elmer) A second scan on the same machine was performed to obtain multi-spectral image acquisition on 10 to 15 fields/slide at 10× magnification. Multi-spectral images were analysed using the Trainable WEKA Segmentation mode using the InForm software (Perkin Elmer).

### Statistical Analysis

All data are expressed as mean±standard deviation (SD). The number of replicates is indicated in each figure and each replicate represent a biological rather than an experimental replicate. Data are analysed and graphs are generated with GraphPad Prism version 8 (GraphPad Software, Inc, USA). Statistic test has been chosen depending on the biological question behind the experiment. Briefly we used Student's t-test, one- or two-way ANOVA followed by an appropriate post-hoc test. The test used is stated in each figure legend. P<0.05 is considered statistically significant.

For all in vitro experiments a two-sided test is considered. All data were tested for normal distribution and equal variance before performing any statistical analysis using Prism v8.

We have performed a power calculation for the number of mice to use in the studies on the chronic CCl₄ model based on the data available from previous studies on the level of ALT (indicating liver damage) at 12 weeks of CCl₄ treatment. We have assumed a mu1 of 100 for 12 week-CCl₄ mice treated with hMDMs and a mu2 of 200 for 12 week-CCl₄ mice treated with vehicle (saline), with a sigma of 50. We have set the power desired at 0.80 assuming a statistical significance at the threshold of 0.05. The power calculation returned an n=6. This is the minimal number of mice used in each experiment. We had no available data at the time of the experiment for the APAP overdose mice treated with hMDMs day5 polarised to AAMs for 24 h. We treated the present experiment as a pilot and we plan to expand the treated cohort in the future.

For all in vivo experiments a one-sided test is considered, as we are testing the hypothesis that human macrophages reduce necrosis and fibrosis in APAP overdose and CCl₄ models, respectively. All data were tested for normal distribution and equal variance before performing any statistical analysis using Prism v8. Specific tests used are indicated in each legend to figure. Power calculation has been performed using the free online tool available at http://www.stat.ubc.ca.

### Results

The inventive differentiation protocol delivers an increase in yield and viability of macrophages as compared to the current gold standard day7 protocol

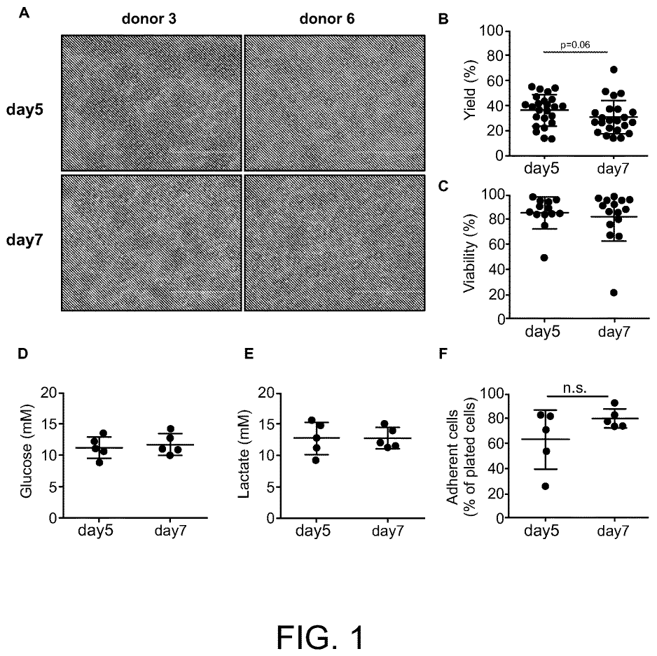

Monocytes cultured for five days (herein referred to as day5) in TexMacs medium with 100 ng/mL show similar characteristics to those cultured for seven days with a re-feed at day three (current gold standard protocol, herein referred to as day7) as observed using brightfield microscopy (FIG. 1A). The day5 protocol shows a strong trend towards a better yield (FIG. 1B, p=0.06), similar viability and metabolism (FIG. 1C-E) as compared to the day7 protocol. One of the main concerns in delivering cell therapy by i.v. infusion is the potential for cells to clump once in the bloodstream. We tested whether the day5 protocol raised the ability of the hMDMs to adhere to a plastic surface. We observed that the day5 hMDMs show a trend towards less adhesion (FIG. 1F, p=0.1875) as compared to the day7 hMDMs.

The inventive differentiation protocol leads to the production of mature, fully functional GMP-compliant macrophages

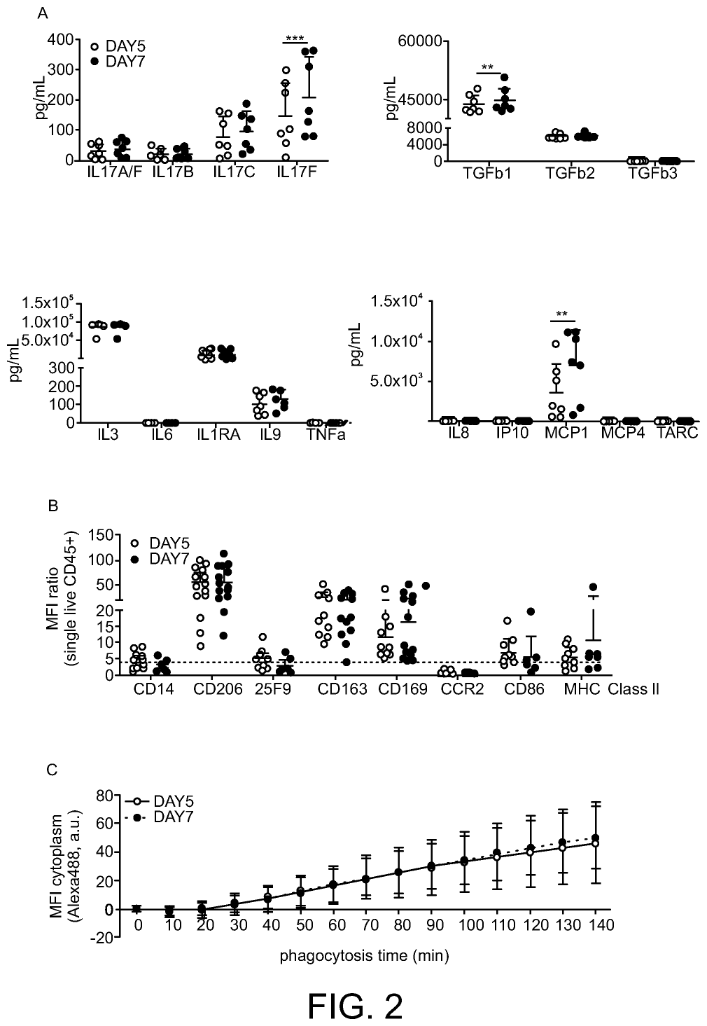

A major concern in shortening the protocol and eliminating the feed is the generation of partially mature, non-fully functional macrophages. We compared the secretion profile of the day5 vs. day7 hMDMs using a highly sensitive 54-plex platform to test the cell culture supernatants at the end of the differentiation protocol. We report herein the results of the dosage of some cytokines, chemokines and growth factors involved in acute and chronic liver disease (FIG. 2A) (a correction factor that takes into account differences in time of culture and yield has been applied). Day5 hMDMs show reduced levels of IL17F and TGFβ1 (Transforming Growth Factor β1), both previously associated with a detrimental effect during liver cirrhosis (21-24). Day5 hMDMs do not upregulate cytokines such as IL6, IL8 and TNFα (Tumour Necrosis Factor α), thereby confirming that these cells are safe to be used as cell therapy as they are unlikely to cause a cytokine storm upon injection (see also Table 5).

A major issue with using a shorter protocol without re-feeding could be the generation of hMDMs lacking some of the cell surface receptors typical of mature macrophages, such as CD206 (Mannose Receptor), CD163 (Hemoglobin-Aptoglobin Scavenger Receptor) and CD169 (sialoadhesin). Macrophages generated with a shorter protocol may also have aberrant expression of antigen presenting molecule (e.g. CD86 and MHC Class II) and may fail to downregulate CCR2 (C-C Chemokine Receptor type 2, or CD192). We show herein that day5 hMDMs have a cell surface marker expression similar to day7 hMDMs (FIG. 2B and Table 1). Currently, the minimal standard to define a ‘mature’ macrophage generated for clinical use (as per MHRA-compliant clinical protocol) is a 5-fold increase in the expression of CD206 and 25F9 (a marker associated with macrophage maturation). Both our protocols meet this condition (dotted line in FIG. 2B). 25F9 is not strongly upregulated in our preparations as compared to the currently used clinical product because our starting material consists of monocytes from peripheral blood mononuclear cell preparations taken from healthy donor blood donations, which have a higher 25F9 expression than monocytes from leukapheresis (i.e. those used to generate the day7 product for clinical use).

One of the desired function of macrophages once transferred in a patient is phagocytosis: Phagocytosis contributes to dead or dying cell clearance, restoration of the liver's barrier function against bacteria of gut origin, and conversion of macrophage phenotype from pro-inflammatory to pro-restorative. In fact, one of the main concerns in reducing the cell culture time and eliminating the feeding is that the hMDMs obtained could be less efficient at performing phagocytosis. Using a live imaging approach to measure phagocytosis of zymosan A-coated beads by day5 vs day7 hMDMs we demonstrated that the hMDMs generated with the two protocols are comparable in terms of phagocytic capacity (FIG. 2C and Table 4).

TABLE 3
RFI of cell surface markers in hMDMs day5 no-feed and day7 standard protocol

| marker | DAY5 av | DAY5 SD | n | DAY7 av | DAY7 SD | n |
|---|---|---|---|---|---|---|
| CD14 | 3.526405 | 2.236156 | 18 | 2.044955 | 1.316308 | 14 |
| CD206 | 54.95115 | 28.16548 | 18 | 55.68434 | 31.11817 | 14 |
| 25F9 | 4.20196 | 2.513642 | 18 | 2.697895 | 1.591471 | 14 |
| CD163 | 23.63787 | 11.53435 | 13 | 20.3617 | 9.779279 | 13 |
| CD169 | 11.61222 | 9.765346 | 13 | 16.2687 | 14.03805 | 13 |
| CCR2 | 0.232945 | 0.2091234 | 13 | 0.2619833 | 0.1339491 | 13 |
| CD86 | 6.808384 | 4.273312 | 10 | 5.580 | 6.354599 | 7 |
| MHC Class II | 5.21494 | 3.227986 | 10 | 10.47571 | 16.34271 | 7 |

TABLE 4
Alex488 cytoplasmic MFI as a measure of phagocytosis in day5 no-feed and day7 standard
protocol hMDMs

| | DAY5 | | | DAY7 | | |
|---|---|---|---|---|---|---|
| TIME | MEAN | SD | n | MEAN | SD | n |
| 0 | 0.000 | 0.000 | 7 | 0.000 | 0.000 | 6 |
| 10 | −1.441384 | 2.895393 | 7 | −1.2243 | 4.132898 | 6 |
| 20 | 0.3100007 | 4.042008 | 7 | 0.03003117 | 6.089865 | 6 |
| 30 | 3.872093 | 6.089653 | 7 | 3.304473 | 7.824904 | 6 |
| 40 | 8.268787 | 8.024744 | 7 | 7.496515 | 9.180511 | 6 |
| 50 | 13.12365 | 10.32989 | 7 | 11.64244 | 11.01407 | 6 |
| 60 | 17.68846 | 12.61675 | 7 | 16.20512 | 12.25722 | 6 |
| 70 | 22.09463 | 15.05047 | 7 | 20.88522 | 13.55408 | 6 |
| 80 | 25.95883 | 17.14326 | 7 | 25.60332 | 14.70255 | 6 |
| 90 | 29.56796 | 19.36675 | 7 | 30.29026 | 15.81196 | 6 |
| 100 | 33.09268 | 21.40685 | 7 | 34.29896 | 16.95813 | 6 |
| 110 | 36.85434 | 23.43145 | 7 | 38.40984 | 18.2587 | 6 |
| 120 | 40.00526 | 25.15578 | 7 | 42.76797 | 19.05064 | 6 |
| 130 | 43.19535 | 26.48693 | 7 | 46.53022 | 20.54206 | 6 |
| 140 | 46.29604 | 28.4563 | 7 | 50.02634 | 21.74133 | 6 |

TABLE 5
Secretion profile of day5 (D5) vs. day7 (D7) standard protocol hMDMs:

| | | | | | | t-test or non- |
|---|---|---|---|---|---|---|
| | | D5 | | D7 | | parametric test |
| | | Average | SD | Average | SD | D5 vs D7 |
| IL17 family | IL17A/F | 26.88 | 25.14 | 37.09 | 28.00 | 0.0427 |
| | IL17B | 19.32 | 16.02 | 21.12 | 16.27 | 0.4478 |
| | IL17C | 76.88 | 65.18 | 98.03 | 63.89 | 0.1052 |
| | IL17F | 146.76 | 110.29 | 210.91 | 128.66 | 0.0182 |
| TGFb family | TGFb1 | 43871.69 | 2290.83 | 44755.05 | 3038.96 | 0.0444 |
| | TGFb2 | 6066.80 | 322.57 | 6108.69 | 368.11 | 0.5375 |
| | TGFb3 | 13.06 | 1.84 | 14.64 | 0.76 | 0.0563 |
| Vacsular | SAA | 137.30 | 196.15 | 109.55 | 160.79 | 0.6398 |
| injury | CRP | 712.06 | 499.76 | 629.62 | 204.40 | 0.6499 |
| | sVCAM1 | 6412.58 | 1050.65 | 6887.30 | 1843.64 | 0.2543 |
| | sICAM1 | 30.58 | 18.44 | 17.13 | 11.79 | 0.0651 |
| Angiogenesis | VEGF-A | 183.49 | 126.81 | 135.16 | 131.12 | 0.3665 |
| family | VEGF-D | 0.00 | 0.00 | 3.39 | 5.21 | 0.1360 |
| | PIGF | 0.28 | 0.40 | 0.30 | 0.25 | 0.9102 |
| | bFGF | 0.53 | 0.34 | 0.61 | 0.41 | 0.6795 |
| | VEGFR1 | 124.45 | 75.09 | 171.11 | 102.55 | 0.0109 |
| cytokines | IL3 | 85471.30 | 13505.88 | 86694.60 | 14221.14 | 0.0596 |
| | IL6 | 0.57 | 0.40 | 0.59 | 0.42 | 0.9215 |
| | IL1RA | 10710.74 | 9054.36 | 12113.01 | 10312.49 | 0.2939 |
| | IL9 | 103.70 | 55.62 | 128.67 | 51.26 | 0.0106 |
| | TNFa | 2.54 | 1.73 | 1.87 | 1.06 | 0.0708 |
| chemokines | IL8 | 2.27 | 3.10 | 5.50 | 9.73 | 0.4878 |
| | IP10 | 81.41 | 41.38 | 105.20 | 61.46 | 0.2369 |
| | MCP1 | 3610.78 | 3491.17 | 7055.61 | 4318.41 | 0.0631 |
| | MCP4 | 5.18 | 9.06 | 24.16 | 16.85 | 0.0549 |
| | TARC | 9.54 | 9.66 | 10.29 | 6.34 | 0.8298 |

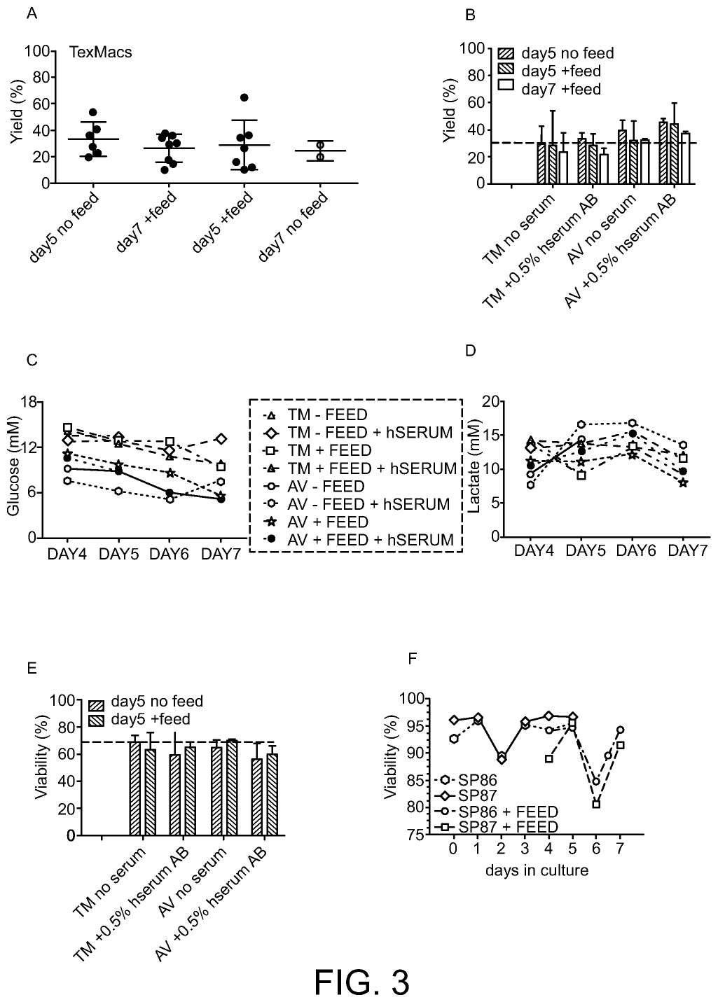

We tested whether the presence of a small percentage of human serum type AB (0.5%) and/or the usage of another cell culture medium (AimV) could further improve yield and viability of day5 hMDMs. As shown in FIG. 3, we failed to identify any differences in yield and viability (FIGS. 3A-B and E). We also observed that AimV-cultured cells were in general more prone to form clumps in cell culture, therefore raising a major safety concern around its use. We concluded that the usage of human serum does not improve yield, viability or metabolism (FIG. 3C-D) of day5 hMDMs. We also decided against the use of AimV in further experiment because of the cell clumping concern.

Characterisation of the day-by-day differentiation of human monocytes into macrophages using the inventive protocol

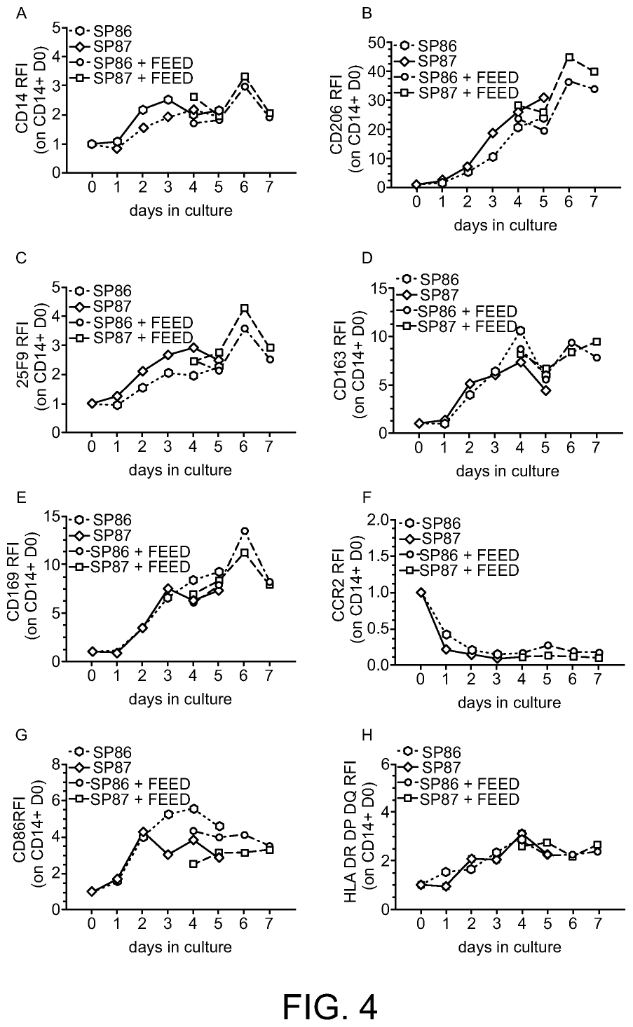

Despite the most common finding in literature is that macrophages need to be cultured for around a week, we have shown that day5 hMDMs are fully mature and functional. We reasoned that perhaps some characteristic of mature macrophages could be present even before the fifth day of culture. We therefore performed a day-by-day flow cytometry of cell surface markers on monocytes from two distinct donors (FIG. 4). We cultured the monocytes for either five or seven days, with or without feeding. We stopped the analysis of the unfed cells at day5. We were able to show that CCR2 is downregulated as early as day one after the start of the culture, and it is at levels similar to those of mature macrophages from day 2 onwards (FIG. 4F). Markers such as CD206, 25F9 and CD169 increase over time, and peak at day 6, to then decline by day 7 (FIG. 4B, C, E). An upregulation of these markers are seen as early as 3 days in culture (FIG. 4B, C, E).This reinforces our hypothesis that day7 hMDMs maybe be too “spent” to perform optimal therapeutic functions once transferred in vivo. Our analysis also unveiled that hMDMs cultured in TexMacs supplemented with 100 ng/mL MCSF have a cell surface marker expression compatible with mature cells from day 4, regardless of the presence or absence of feeding. However, the expression of some of the cell surface marker at day 4 is not stabilised yet as shown by the variable levels of CD163 (FIG. 4D) and by fluctuating levels of CD86 (FIG. 4G). The present inventors have identified that cells at day 4 are juvenile cells and therefore more amenable to processing such as cryopreservation and transfection. The analysis of the viability during the cell culture reveal a drop at day2 and day6 (FIG. 4E). The drop at day2 explains the lower-than-100% yield that we routinely observe in our experiments. The drop at day6 could justify the trend to a much lower yield in the day7 vs. day5 protocol, as reported in FIG. 1B.

### The Macrophages of the Invention are Mature and Able to Respond to Inflammatory Cues

One of the key features of macrophages is their ability to respond to inflammatory cues. It has long been described that in vivo macrophages are a highly heterogeneous population, which acquire distinct polarisation depending on the microenvironment. Hyper-responsiveness to inflammatory cues is one of the concerns when using a day5 vs a day 7 hMDMs differentiation protocol. We therefore went on to demonstrate that our day5 hMDMs is safe and does not generate cells that could potentially cause a cytokine storm once injected in a patient with an ongoing acute or chronic liver inflammation.

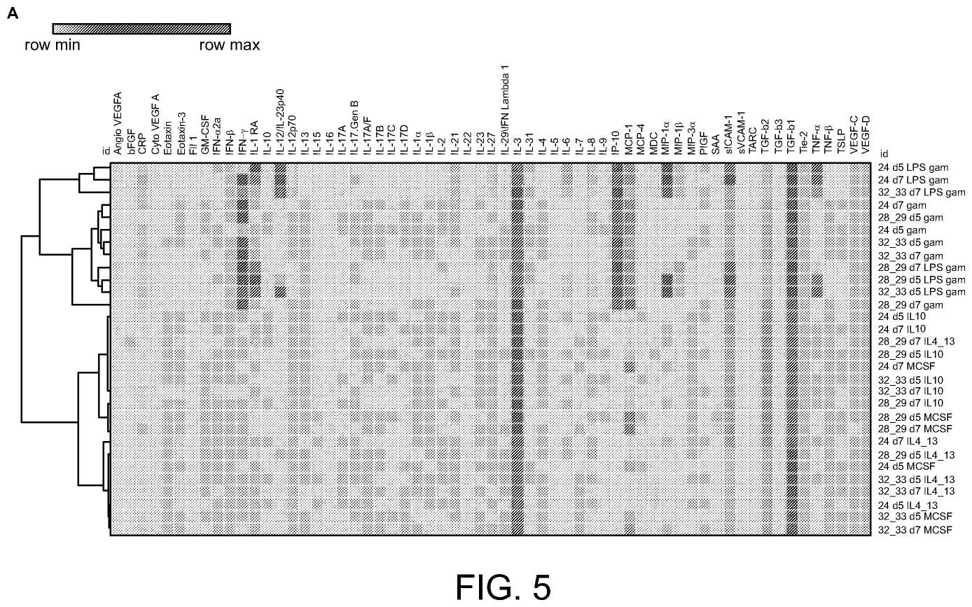

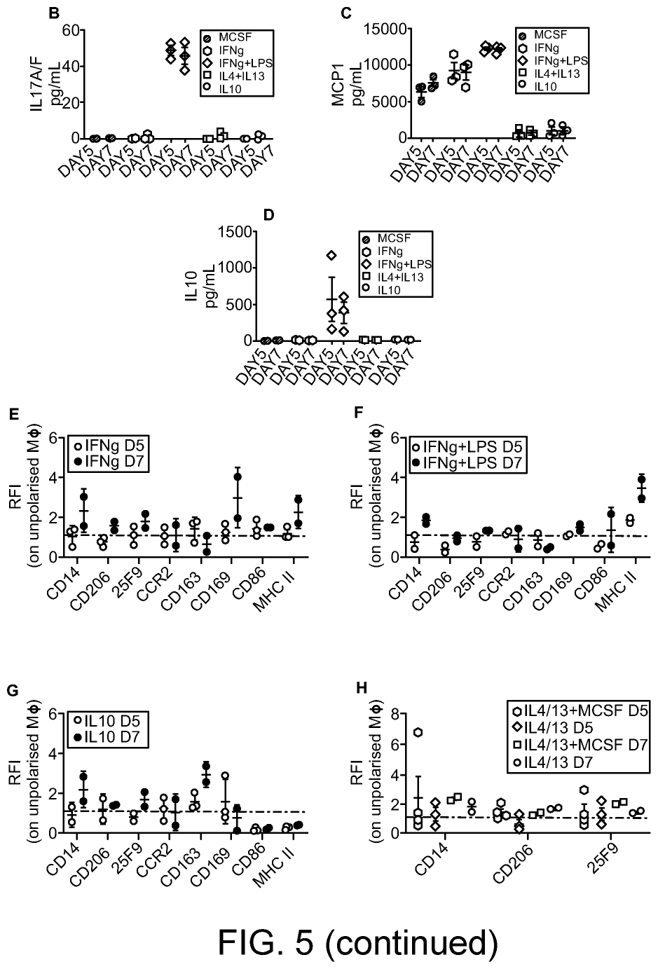

To this end, we collected supernatants from day5 hMDMs and day7 hMDMs after 24 h stimulation with IFNγ, IFNγ plus LPS, IL4 plus IL13 and IL10. We performed the same 54-plex analysis reported in FIG. 2 followed by a clustering analysis using the online tool Morpheus. Data suggest that IFNγ and IFNγ plus LPS-stimulated macrophages clustered together, regardless of whether the unpolarised macrophages were differentiated using a day5 or day7 protocol. Interestingly, IL4 plus IL13 and IL10 macrophages, which should share more anti-inflammatory, pro-restorative properties, clustered together based on the donor they came from, rather than the cell culture protocol to obtain the unpolarised macrophages (FIG. 5A). A closer look at some important cytokines and chemokines in acute and chronic liver disease (IL17A/F, MCP1 and IL10) further confirms that no significant differences are detectable in the way day5 unpolarised macrophages react to inflammatory stimuli (FIG. 5B-D). These results suggest that the day5 hMDMs differentiation protocol leads to the generation of cells that do not over-react to inflammatory cues and are therefore unlikely to cause a cytokine storm once injected in a patient with acute or chronic liver disease.

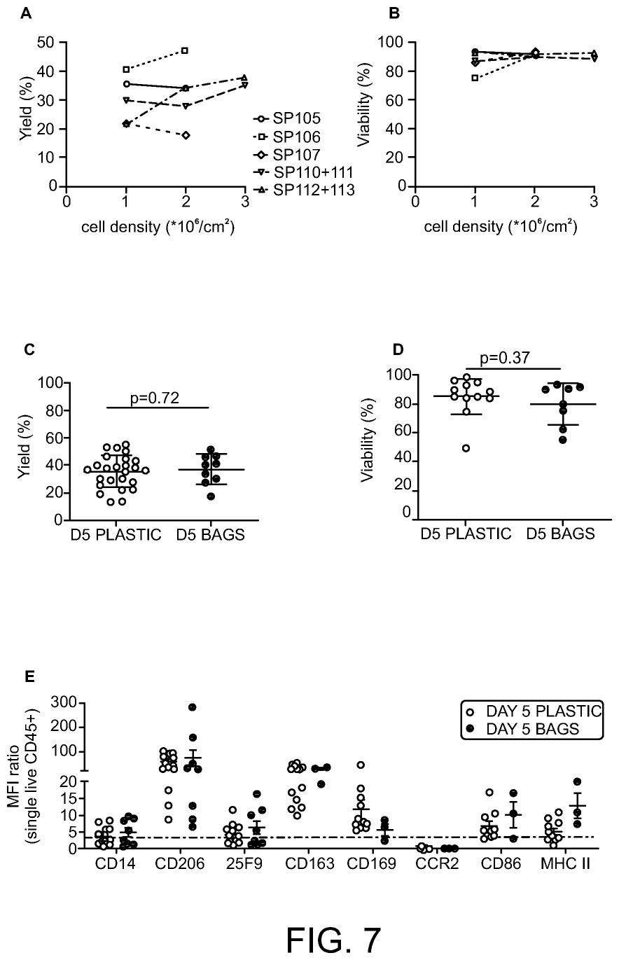

Polarised macrophages can be beneficial in a number of pathological setting, and to favour the success of other cell therapies. Therefore, there could be an interest in producing polarised macrophages starting from the day5 or day7 unpolarised hMDMs. We verified by flow cytometry whether day5 and day7 hMDMs have a similar cell surface marker expression following polarisation. We calculated the RFI by dividing the MFI of the polarised for the MFI of the unpolarised hMDMs: a RFI>1 means upregulation; a RFI<1 means downregulation (FIG. 5E-H). The main feature of LPS-stimulated macrophages is the upregulation of antigen presenting molecule such the MHC II molecules (Major Histocompatibility Complex Class II). Both day7 and day5-polarised hMDMs showed an increase of their MHC II molecules (FIG. 5F). Following stimulation with IFNγ, day7-polarised hMDMs showed an unusual upregulation of CD206, a scavenger receptor that should be induced by the IL4/1L13 stimuli. Conversely, IFNγ day5-polarised hMDMs failed to show this upregulation (FIG. 5E). IL10 stimulation led to the upregulation of CD163 and the downregulation of CD86 and MHCII in both day5 and day7 hMDMs, as expected (FIG. 5G). However, when we used the IL4/1L13 combination we fail to see a significant upregulation of CD206 in day5-polarised hMDMs (FIG. 7A). We reasoned that adding fresh MCSF could have better supported polarisation. We therefore run a comparison between day5 and day7-polarised hMDMs, in the presence or in the absence of MCSF. The addition of MCSF resulted in CD206 upregulation in IL4/1L13 day5 polarised hMDMs (FIG. 5H). These data reinforce the concept that day5 hMDMs are mature macrophages, able to normally respond to inflammatory stimuli, and that can be used as starting material for the production of polarised human macrophages to use as cell therapy.

The Inventive differentiation protocol leads to the production of mature, fully functional macrophages using GMP-graded cell culture bags

hMDMs for clinical use cannot be grown using cell culture-treated plastic. Normally, hMDMs for clinical use are cultured using GMP-compliant cell culture bag. Therefore, we sought to validate our day5 no feed differentiation protocol using the above-mentioned cell culture bag as a support.

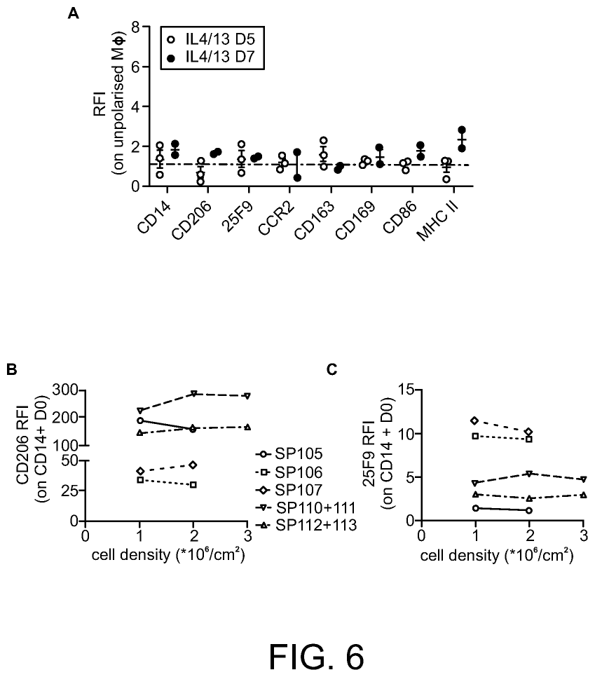

We reasoned that perhaps a critical factor for the growth in GMP-graded cell culture bag is cell density: We tested our day5 hMDMs differentiation protocol using the density used in plastic (2×10⁶/cm²), a lower (1×10⁶/cm²) and a higher (3×10⁶/cm²) density. We also pondered that macrophages may benefit from a more crowded environment at the start of the differentiation process, to then benefit from less dense culture conditions. Therefore, we went on to culture monocyte/macrophages at 2×10⁶/cm² till day3, to then dilute them at 1×10⁶/cm² for the last two days of culture. Best results in terms of yield were obtained using 3×10⁶/cm² (FIG. 7A). Viability was very variable at 1×10⁶/cm². 2×10⁶/cm² and 3×10⁶/cm² cell density showed a consistent viability of 90% or more in all donors analysed (FIG. 7B). We then compared the cell surface expression of mature macrophage markers in day5 hMDMs cultured in cell culture bags at distinct densities. Day5 hMDMs from all donor showed high expression of CD206; only one donor (donor SP105) showed levels of 25F9 non compatible with the current release criteria (RFI≥4). Variation between protocols were minimal (FIG. 6B, C). Diluting the macrophages during the last two days of culture did not offer a significant advantage as compared to the 2×10⁶/cm² cell density (not shown). The slightly worst results on viability obtained with the 1×10⁶/cm² suggest that macrophages need paracrine signals for their differentiation and growth. We concluded that a density of 2×10⁶/cm² or 3×10⁶/cm² can be used in the future for the production of day5 hMDMs for clinical use.

We then ought to confirm that the functional results obtained using hMDMs cultured on plastics were translatable to hMDMs cultured in cell culture bags. To this end we carried on a comparison of the yield, viability and cell surface markers of the day5 hMDMs culture in plastic or GMP-graded cell culture bags at a density of 2×10⁶/cm². Cells cultured in bags show similar yield and viability to those cultured using tissue culture-treated plastic plates (FIG. 7C, D). The expression of cell surface markers is similar between the d5 hMDMs cultured in bags and in plastic (FIG. 7E and Table 6).

TABLE 6
RFI of cell surface markers in hMDMs day5 no feed protocol in GMP vs. standard plastic

| | DAY5 | DAY5 plastic | | DAY5 GMP | DAY5 GMP | |
|---|---|---|---|---|---|---|
| marker | plastic av | SD | n | bag av | bag SD | n |
| CD14 | 3.526405 | 2.236156 | 18 | 4.84125 | 3.85 | 8 |
| CD206 | 54.95115 | 28.16548 | 18 | 71.50875 | 98.17 | 8 |
| 25F9 | 4.20196 | 2.513642 | 18 | 6.3325 | 5.73 | 8 |
| CD163 | 23.63787 | 11.53435 | 13 | 27.24 | 7.420505 | 3 |
| CD169 | 11.61222 | 9.765346 | 13 | 5.753 | 3.05896 | 3 |
| CCR2 | 0.232945 | 0.2091234 | 13 | 0.1467 | 0.040414 | 3 |
| CD86 | 6.808384 | 4.273312 | 10 | 10.11 | 6.819318 | 3 |
| MHC Class II | 5.21494 | 3.227986 | 10 | 12.77 | 6.472328 | 3 |

### Macrophages of the Invention are Safe to Inject in Mouse Models of Acute and Chronic Liver Injury

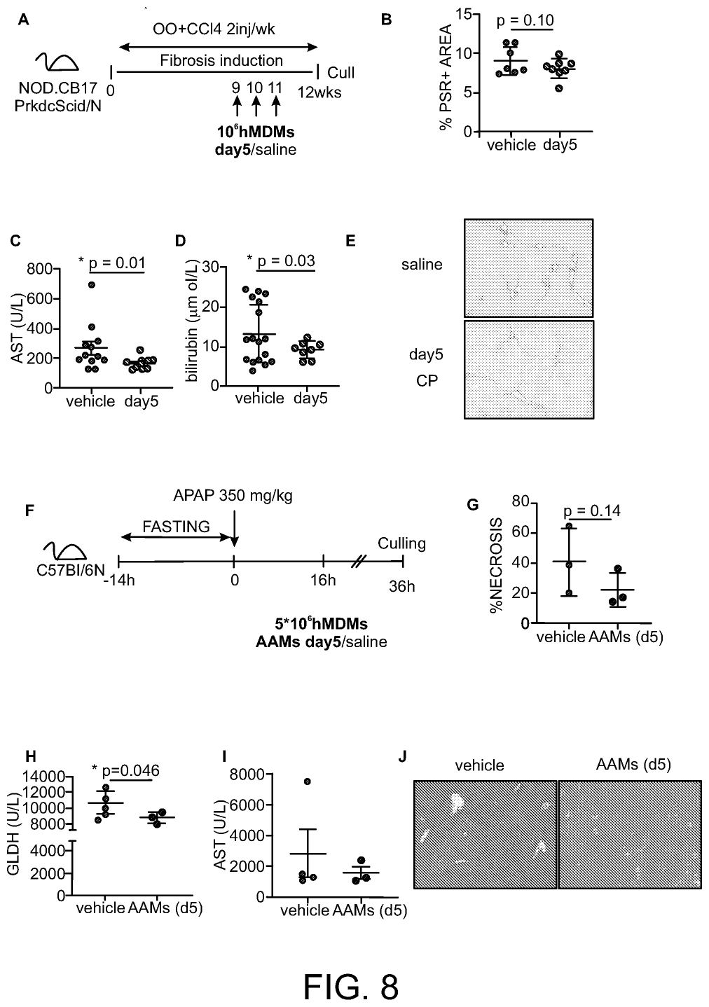

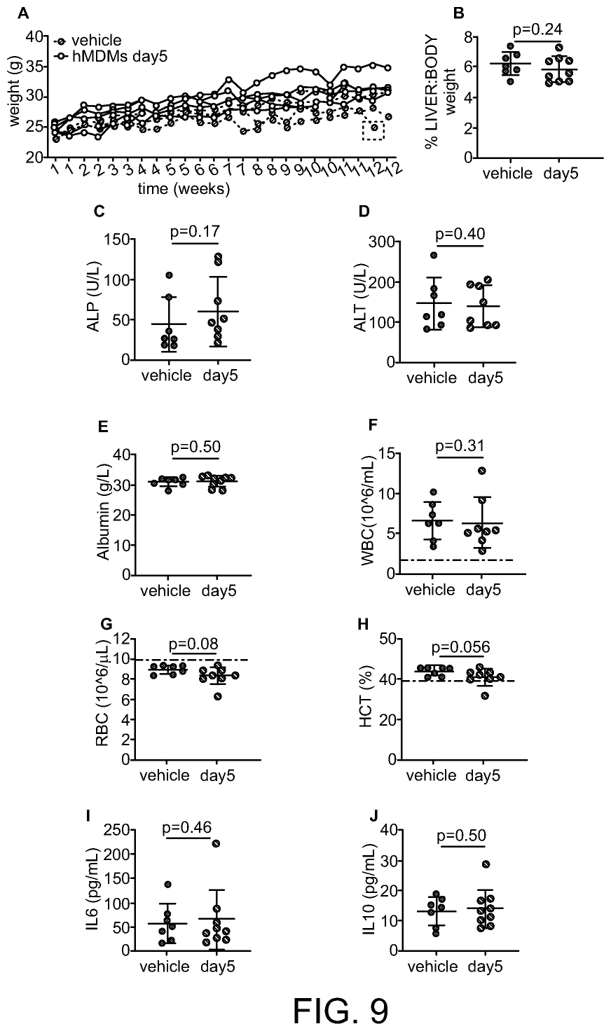

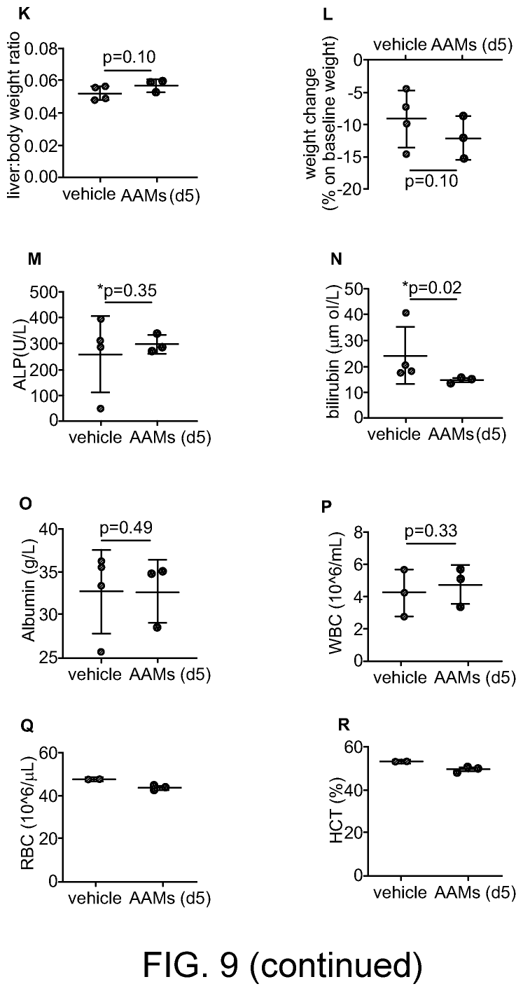

To definitely prove that our day5 hMDMs are a suitable product for cell therapy of acute and chronic liver injury we needed to test their safety in mouse models. We also aimed to show signs of efficacy of the therapy. To this end we induced liver cirrhosis by injecting carbon tetrachloride (CCl₄) into immunodeficient mice twice a week for 12 weeks. We injected 10⁶ day5 hMDMs or vehicle (saline) at the beginning of week 9,10 and 11. We culled the mice one week later (FIG. 8A). The mice did not show any adverse event at the time of injection of the cell therapy, nor during the week prior to culling. Mice in the treated group did not lose weight and their weight trends over time and liver:body weight ratio at cull were comparable to controls (FIG. 9A, B). Haematological parameters such as white blood cells (WBC), red blood cells (RBC) and haematocrit (HCT) were comparable in the two groups of mice, too (FIG. 9F, G, H), thereby excluding severe adverse effects on the bone marrow of the treated mice. There is a significant trend to have a reduce HCT in the treated mice, a trend potentially relevant as the therapy restores normal HCT levels for adult male NOD/SCID mice (pink dashed line in FIG. 9H). Cytokines such as IL6 and IL10 were at comparable levels in the plasma of the two groups of mice (FIG. 9I, J), confirming that the day5 hMDMs are safe to inject and do not trigger a cytokine storm when injected in a mouse experiencing chronic inflammation.

Further, we assessed whether we could detect signs of efficacy. To this end we performed liver function tests (LFTs) on the sera of treated and control mice. No difference in the levels of ALT was detected, although we reported a trend do decreased AST (FIG. 8C, FIG. 9D). No differences in the levels of ALP and albumin were detected either (FIG. 9C, E). Remarkably, bilirubin was significantly reduced in the sera of mice treated with hMDMs day5 (FIG. 8D). ALT and AST circulating levels can be influenced by various factors, including macrophage phagocytosis: in fact, they can be internalised by macrophages via this process. Therefore, an analysis of fibrosis in the liver tissue is needed to assess efficacy. We performed picrosirius red (PSR) staining on the liver sections from the treated and the control group to quantify fibrosis: treated mice showed a strong trend towards reduced fibrosis (FIG. 8B, E). Immunodeficient mice lack the adaptive arm of immunity, a key player in liver regeneration. They are therefore partially impaired in taking advantage of the paracrine effect of macrophage cell therapy. Furthermore, the immune response to damage is not the same magnitude of wild type mice, thereby partially impairing the microenvironment-macrophage cross-talk, a process crucial to mediate the therapeutic effect of macrophages. Therefore, a trend towards reduction both in circulating AST and liver fibrosis, and a significant reduction in circulating bilirubin is a remarkable result. In conclusion, data suggest that injecting day5 hMDMs in models of chronic liver disease is safe and partially efficacious in reducing liver fibrosis.

Previous work in the lab suggested that alternatively activated macrophages (IL4/IL13 polarised, herein referred to as AAMs) are useful to contain acute liver injury by paracetamol (acetaminophen, APAP) overdose and to promote liver regeneration in mice (Starkey Lewis P J et al., J Hep, 2020). We sought to repeat the same data using our day5 hMDMs polarised using IL4/IL13 plus MCSF, as described in FIG. 7 as product for cell therapy of APAP overdose. APAP overdose was induced in C57Bl/6 immunocompetent mice (FIG. 8F). We previously demonstrated that healthy immunocompetent mice receiving day7 hMDMs do not show signs of rejection or toxicity up to one week after the injection of the cells (not shown). This prompted us to use an immunocompetent mouse for our safety and efficacy test as this overcomes the limitations of immunocompromised mice listed above. The treated mice showed a trend towards an increased liver:body weight ratio and increased weight loss (FIG. 9K, L), which could potentially raise safety concerns. However, haematological parameters such as WBC, RBC and HCT were comparable in the two groups of mice (AAMs day5 treated vs. vehicle treated) (FIG. 9P, Q, R), thereby excluding an ongoing toxicity at bone marrow level. Further, ALP and Albumin were comparable in the two groups of mice (FIG. 9M, O), excluding an exacerbation of the liver damage by the AAMs day5 therapy. Conversely, mice showed significantly reduced levels of circulating GLDH (FIG. 8H), a sensitive marker for necrotic tissue damage. Mice also showed smaller necrotic area when treated with AAMs day5 as compared to livers of untreated mice, as assessed by image quantification of H&E sections (FIG. 8G, J). A further indication of efficacy is given by the reduction in circulating bilirubin observed in the treated mice (FIG. 9N).

In conclusion, our data shows that we have set up a protocol for generating GMP-graded hMDMs in a cheaper and faster way as compared to current standard. This result has been achieved by combining two approaches: reducing the cell culture time and eliminating the feeding step. The use of the optimal cell density, a serum-free T-cell medium, with the support of rMCSF, guarantees a product that has superior qualities to the day7 hMDMs; the current gold standard in the field. In particular, day5 hMDMs shows a less pro-inflammatory and less pro-fibrogenic secretion profile (lower levels of IL17F and TGFβ in the cell culture supernatants respectively). Despite literature in the field suggesting that day7 is the optimal length of culture to produce mature hMDMs, we showed that our day5 hMDMs product is comparable in terms of cell surface marker expression and phagocytosis to the day7 hMDMs product. Furthermore, the day5 hMDMs differentiation protocol shows a better yield as compared to the day7 hMDMs protocol. Day5 hMDMs furthermore proved safe and efficacious in mouse models of APAP overdose (acute liver injury); they also proved safe and partially efficacious in mouse models of liver cirrhosis (chronic liver injury).

In view of the above, it will be appreciated that the present disclosure also relates to the following clauses:

Clause 1: An in vitro method of producing macrophages comprising:
(a) Culturing monocytes in medium for 4-5 days to produce macrophages, wherein the medium comprises one or more growth factors to stimulate macrophage production;

And wherein step (a) takes place entirely in the same medium.

Clause 2: The method according to clause 1, wherein the monocytes are cultured for 5 days.

Clause 3: The method according to any one of clauses 1 to 2, wherein the method does not comprise re-feeding or replacing medium.

Clause 4: The method according to any one of clauses 1 to 3, wherein the monocytes are seeded at a density of between 1×10⁶ cells/cm² up to 1×10⁷ cells/cm².

Clause 5: The method according to any one of clauses 1 to 4, wherein the medium is selected from X-Vivo 10, TexMACS, AIMv, RPMI, DMEM, and DMEM/F12.

Clause 6: The method according to clause 5 wherein the medium is a T-cell medium, preferably TexMACS.

Clause 7: The method according to any one of clauses 1 to 6, wherein the medium comprises one or more factors selected from the CSF family, preferably M-CSF.

Clause 8: The method according to any one of clauses 1 to 7, wherein the medium contains M-CSF at a concentration of between 25-150 ng/mL.

Clause 9: The method according to any one of clauses 1 to 8, wherein the monocytes are human, and the macrophages are human monocyte derived macrophages (hMDMs).

Clause 10: The method according to any one of clauses 1 to 9, wherein the monocytes are derived from human blood, preferably the buffy coat of human blood, preferably from the PBMC fraction of human blood.

Clause 11: The method according to any one of clauses 1 to 10, wherein the method further comprises a step of polarisation of the macrophages produced in step (a), preferably into M1-like or M2-like macrophages.

Clause 12: The method according to clause 11, wherein polarising factors are added to the medium, preferably M1 or M2 polarising factors.

Clause 13: The method according to clause 12, wherein the M1 polarizing factors are selected from: GM-CSF, IFNγ, and TLR agonists such as LPS; and the M2 polarizing factors are selected from: IL10, IL4, IL13, and poly(I:C).

Clause 14: The method according to any one of clauses 1 to 13, wherein the method is GMP-compliant.

Clause 15: The method according to one of clauses 1 to 14, wherein the yield of mature macrophages is at least 30%, at least 35%, at least 40%, at least 45%, at least 50%, at least 55%, at least 60%, at least 65%, at least 70%, at least 75%, at least 80%.

Clause 16: A macrophage produced by the method according to any of one of clauses 1 to 15.

Clause 17: An ex vivo generated macrophage having a pro-regenerative phenotype.

Clause 18: The macrophage according to any one of clauses 16 or 17, wherein the macrophage has increased expression of one or more pro-regenerative cytokines.

Clause 19: The macrophage according to any of one clauses 16 to 18, wherein the macrophage has increased expression of one or more anti-inflammatory cytokines and/or reduced expression of one or more inflammatory cytokines, and/or increased expression of one or more anti-fibrogenic cytokines, and/or reduced expression of one or more fibrogenic cytokines.

Clause 20: The macrophage according to any of one clauses 16 to 19 wherein the macrophage has reduced expression of one or more of the following cytokines: IL1, IL12, IL17 (A, B, C, F), IL18, TNFα, IFNγ, preferably IL17F.

Clause 21: The macrophage according to any of one clauses 16 to 20, wherein the macrophage has reduced expression of one or more of the following cytokines: IL4, IL13, PDGF, TGFβ (1, 2, 3), preferably TGFβ1.

Clause 22: The macrophage according to any of one clauses 16 to 21, wherein the macrophage expresses mature cell surface markers, preferably CCR2−, CD14+, CD206+, CD163+, CD169+, 25F9+, and CD86+.

Clause 23: A population of macrophages according to any of one clauses 16 to 22.

Clause 24: A composition comprising a population of macrophages according to clause 23.

Clause 25: A macrophage, population or composition according to any of one clauses 16 to 24 for use as a medicament.

Clause 26: A macrophage, population, or composition according to any of one clauses 16 to 24 for use in the treatment of liver disease or injury.

Clause 27: A macrophage, population, or composition for use according to clause 26, wherein the liver disease is acute or chronic.

Clause 28: A macrophage, population or composition for use according to clause 27, wherein the acute liver disease is a drug overdose preferably APAP overdose, and wherein the chronic liver disease is liver cirrhosis.

Clause 29: A population or composition according to clause 23 or 24, wherein the macrophages express an average of less than 200 pg/mL of IL17F, preferably less than 190 pg/mL of IL17F, preferably less than 180 pg/mL of IL17F, preferably less than 170 pg/mL of IL17F, preferably less than 160 pg/mL of IL17F, preferably 150 pg/mL or less of IL17F.

Clause 30: A population or composition according to clauses 23, 24 or 29, wherein the macrophages express an average of less than 45000 pg/mL of TGFβ, preferably less than 44000 pg/mL, preferably less than 43000 pg/mL, preferably less than 42000 pg/mL, preferably less than 41000 pg/mL, preferably 40000 pg/mL or less of TGFβ.

Clause 31: A population or composition according to clauses 23, 24, 29 or 30, wherein the macrophages express on average more than 50 pg/mL VEGFR1, preferably more than 100 pg/mL of VEGFR1, preferably more than 120 pg/mL of VEGFR1, preferably more than 140 pg/mL VEGFR1, preferably more than 160 pg/mL VEGFR1, preferably more than 170 pg/mL of VEGFR1.

Clause 32: A population or composition according to any of clauses 23, 24 and 29-31, wherein the macrophages express on average less than 500 pg/mL of IL9, preferably less than 300 pg/mL of IL9, preferably less than 200 pg/mL IL9, preferably less than 180 pg/mL IL9, preferably less than 160 pg/mL IL9, preferably less than 140 pg/mL IL9, preferably less than 130 pg/mL of IL9.

Clause 33: A population or composition according to any of clauses 23, 24 and 29-32, wherein the macrophages possess an anti-inflammatory and anti-fibrogenic phenotype, wherein the macrophages express on average less than 200 pg/mL of IL17F and less than 45000 pg/mL of TGFβ.

Clause 34: A population or composition according to any one of clauses 23, 24 and 29-33 in which the macrophages have an anti-inflammatory and anti-fibrogenic phenotype, wherein the macrophages express on average around 150 pg/mL of IL17F and around 40000 pg/mL of TGFβ.

Clause 35: A macrophage, population or composition according to any of clauses 16-24 which respond to inflammatory stimuli such as one or more of: IFNγ, IL10, IL4, IL13, and LPS.

Clause 36: A cell culture bag comprising a macrophage of any of clauses 1-22, a population according to clause 23, or a composition according to clause 24.

### Bibliography

1. L. Campana, J. P. Iredale, Regression of Liver Fibrosis. Semin Liver Dis 37, 1-10 (2017).
2. A. Pellicoro, P. Ramachandran, J. P. Iredale, J. A. Fallowfield, Liver fibrosis and repair: immune regulation of wound healing in a solid organ. Nat Rev Immunol 14, 181-194 (2014).
3. E. J. Williams, J. P. Iredale, Liver cirrhosis. Postgrad Med J 74, 193-202 (1998).
4. S. J. Forbes, P. N. Newsome, Liver regeneration—mechanisms and models to clinical application. Nat Rev Gastroenterol Hepatol, (2016).
5. J. A. Thomas, P. Ramachandran, S. J. Forbes, Studies of macrophage therapy for cirrhosis—From mice to men. J Hepatol 68, 1090-1091 (2018).
6. S. J. Forbes, S. Gupta, A. Dhawan, Cell therapy for liver disease: From liver transplantation to cell factory. J Hepatol 62, S157-169 (2015).
7. L. Falasca, A. Bergamini, A. Serafino, C. Balabaud, L. Dini, Human Kupffer cell recognition and phagocytosis of apoptotic peripheral blood lymphocytes. Exp Cell Res 224, 152-162 (1996).
8. P. Ramachandran et al., Differential Ly-6C expression identifies the recruited macrophage phenotype, which orchestrates the regression of murine liver fibrosis. Proc Natl Acad Sci USA 109, E3186-3195 (2012).
9. T. A. Wynn, L. Barron, Macrophages: master regulators of inflammation and fibrosis. Semin Liver Dis 30, 245-257 (2010).
10. E. Zigmond et al., Infiltrating monocyte-derived macrophages and resident kupffer cells display different ontogeny and functions in acute liver injury. J Immunol 193, 344-353 (2014).
11. J. A. Thomas et al., Macrophage therapy for murine liver fibrosis recruits host effector cells improving fibrosis, regeneration, and function. Hepatology 53, 2003-2015 (2011).
12. J. K. Moore et al., Phenotypic and functional characterization of macrophages with therapeutic potential generated from human cirrhotic monocytes in a cohort study. Cytotherapy 17, 1604-1616 (2015).
13. A. R. Fraser et al., Development, functional characterization and validation of methodology for GMP-compliant manufacture of phagocytic macrophages: A novel cellular therapeutic for liver cirrhosis. Cytotherapy 19, 1113-1124 (2017).
14. F. Moroni et al., Safety profile of autologous macrophage therapy for liver cirrhosis. Nat Med, (2019).
15. Q. Wang et al., Interleukin-12 inhibits the hepatocellular carcinoma growth by inducing macrophage polarization to the M1-like phenotype through downregulation of Stat-3. Mol Cell Biochem 415, 157-168 (2016).
16. P. Wang et al., Promising Therapy Candidates for Liver Fibrosis. Front Physiol 7, 47 (2016).
17. F. Tacke, Targeting hepatic macrophages to treat liver diseases. J Hepatol 66, 1300-1312 (2017).
18. S. Friedman et al., Efficacy and safety study of cenicriviroc for the treatment of non-alcoholic steatohepatitis in adult subjects with liver fibrosis: CENTAUR Phase 2b study design. Contemp Clin Trials 47, 356-365 (2016).
19. E. Lefebvre et al., Antifibrotic Effects of the Dual CCR2/CCR5 Antagonist Cenicriviroc in Animal Models of Liver and Kidney Fibrosis. PLoS One 11, e0158156 (2016).
20. F. C. Pearlman, R. T. Lee, Detection and measurement of total bilirubin in serum, with use of surfactants as solubilizing agents. Clin Chem 20, 447-453 (1974).
21. F. C. Paquissi, Immunity and Fibrogenesis: The Role of Th17/IL-17 Axis in HBV and HCV-induced Chronic Hepatitis and Progression to Cirrhosis. Front Immunol 8, 1195 (2017).
22. T. Fabre et al., Type 3 cytokines IL-17A and IL-22 drive TGF-beta-dependent liver fibrosis. Sci Immunol 3, (2018).
23. M. F. Molina, M. N. Abdelnabi, T. Fabre, N. H. Shoukry, Type 3 cytokines in liver fibrosis and liver cancer. Cytokine, (2018).
24. F. Meng et al., Interleukin-17 signaling in inflammatory, Kupffer cells, and hepatic stellate cells exacerbates liver fibrosis in mice. Gastroenterology 143, 765-776 e763 (2012).

### EQUIVALENTS

Those skilled in the art will recognise, or be able to ascertain using no more than routine experimentation, equivalents of the specific embodiments described herein. Such equivalents are intended to be encompassed by the following claims. Any combination of the embodiments disclosed in the any plurality of the dependent claims or Examples is contemplated to be within the scope of the disclosure.

### INCORPORATION BY REFERENCE

The disclosure of each and every patent, patent application publication, and scientific publication referred to herein is specifically incorporated herein by reference in its entirety, as are the contents of its Figures.

1. An in vitro GMP-compliant method of producing macrophages comprising:
(a) culturing monocytes in medium for 3-5 days to produce macrophages, wherein the medium comprises one or more growth factors to stimulate macrophage production;
and wherein step (a) takes place entirely in the same medium.

2. The method according to claim 1, wherein the monocytes are cultured for 5 days.

3. The method according to any preceding claim, wherein the method does not comprise re-feeding or replacing medium.

4. The method according to any preceding claim wherein the monocytes are seeded at a density of between 1×10⁶ cells/cm² up to 1×10⁷ cells/cm².

5. The method according to any preceding claim, wherein the medium is selected from X-Vivo 10, X-Vivo15, TexMACS, AIMv, RPMI, DMEM, and DMEM/F12, preferably TexMACS.

6. The method according to any preceding claim wherein the medium comprises one or more factors selected from the CSF family, preferably M-CSF.

7. The method according to any preceding claim, wherein the medium contains M-CSF at a concentration of between 25-200 ng/mL.

8. The method according to any preceding claim wherein the monocytes are human, and the macrophages are human monocyte derived macrophages (hMDMs).

9. The method according to any preceding claim wherein the monocytes are derived from human blood, preferably the buffy coat of human blood, preferably from the PBMC fraction of human blood.

10. The method according to any preceding claim, wherein the method further comprises a step of polarisation of the macrophages produced in step (a), preferably into M1-like or M2-like macrophages.

11. The method according to claim 10, wherein the further step of polarisation of the macrophages comprises a step of polarising factors added to the medium, preferably M1 or M2 polarising factors.

12. The method according to claim 11, wherein the M1 polarizing factors are selected from: GM-CSF, IFNγ, and TLR agonists such as LPS; and the M2 polarizing factors are selected from: IL10, IL4, IL13, and poly(I:C).

13. The method according to any preceding claim, wherein the method produces mature macrophages with a yield of at least 20%, at least 21%, at least 22%, at least 23%, at least 24%, at least 25%, at least 26%, at least 27%, at least 28%, at least 29%,least 30%, at least 35%, at least 40%, at least 45%, at least 50%, at least 55%, at least 60%, at least 65%, at least 70%, at least 75%, or at least 80%.

14. A macrophage produced by the method according to any of claims 1-13.

15. An ex vivo generated macrophage having a pro-regenerative phenotype, optionally produced by the method according to any of claim 1-13.

16. The macrophage according to claim 14 or 15, wherein the macrophage has increased expression of one or more pro-regenerative cytokines.

17. The macrophage according to any of claims 14-16, wherein the macrophage has increased expression of one or more anti-inflammatory cytokines and/or reduced expression of one or more inflammatory cytokines, and/or increased expression of one or more anti-fibrogenic cytokines, and/or reduced expression of one or more fibrogenic cytokines.

18. The macrophage according to any of claims 14-17 wherein the macrophage has reduced expression of one or more of the following cytokines: IL1, IL12, IL17 (A, B, C, F), IL18, TNFα, IFNγ, preferably IL17F.

19. The macrophage according to any of claims 14-18, wherein the macrophage has reduced expression of one or more of the following cytokines: IL4, IL13, PDGF, TGFβ (1, 2, 3), preferably TGFβ1.

20. The macrophage according to any of claims 14-19 wherein the macrophage expresses mature cell surface markers, preferably CCR2−, CD14+, CD206+, CD163+, CD169+, 25F9+, and CD86+.

21. A population of macrophages according to any of claims 14-20.

22. A composition comprising a population of macrophages according to claim 21.

23. A macrophage, population or composition according to any of claims 14-22 for use as a medicament.

24. A macrophage, population, or composition according to any of claims 14-22 for use in the treatment of disease or injury, wherein the disease is selected from the list comprising liver disease, kidney disease, lung disease or muscle disease.

25. A macrophage, population, or composition for use according to claim 24, wherein the liver disease, kidney disease, lung disease or muscle disease is a fibrotic disease or an inflammatory disease, optionally the disease is acute or chronic, optionally selected from the list comprising a drug overdose preferably APAP overdose and liver cirrhosis.

26. A macrophage, population or composition according to any of claims 16-24 which respond to inflammatory stimuli such as one or more of: IFNγ, IL10, IL4, IL13, and LPS.
